(19) United States
(12) Patent Application Publication
(10) Pub. No.: US 2011/0182880 A1
(43) Pub. Date: Jul. 28, 2011

(54) Combination Therapies Against Cancer

(76) Inventors: Oliver Von Stein, Upplands Vasby (SE); Arezou Zargari, Solna (SE); Åsa Karlsson, Sollentuna (SE); Petra Von Stein, Upplands Vasby (SE); Nikoli Kouznetsov, Jarfalla (SE)

(21) Appl. No.: 12/999,395

(22) PCT Filed: Jun. 18, 2009
(86) PCT No.: PCT/SE2009/050771

§ 371 Date: Mar. 10, 2011

Related U.S. Application Data
(60) Provisional application No. 61/073,664, filed on Jun. 18, 2008.

(51) Int. Cl. A61K 39/395; C07H 21/00; A61K 31/711; A61K 38/22; A61P 35/00

(52) U.S. Cl. 424/130.1; 536/23.1; 514/44 R; 514/9.7

(57) ABSTRACT

Specific oligonucleotide sequences, when given subcutaneously and in particular when administered on a mucous membrane, e.g. intranasally, intravaginally, or rectally, have a profound effect on various human cancer forms as confirmed in vivo, in animal studies, and in vitro, in human PBMCs collected from blood from healthy subjects and from patients suffering from CLL. The compounds are also preferably used in combination with a cancer therapy chosen among radiation treatment, hormone treatment, surgical intervention, chemotherapy, immunological therapies, photodynamic therapy, laser therapy, hyperthermia, cryotherapy, angiogenesis inhibition, or a combination of any of these, and most preferably an immunological treatment comprising the administration of an antibody to the patient.

### TECHNICAL FIELD

The present application relates to the field of medicine, and in particular to novel compounds and methods for use in the treatment of cancer either alone or in combination with existing and future therapies.

### BACKGROUND ART

Cancer treatment has entered an era of targeted approaches. One such approach is use of the immune system to recognize and eliminate malignant cells. Synthetic CpG oligonucleotides (CpG DNA) are a relatively new class of agents that have the ability to stimulate a potent, orchestrated tumour-specific immune response (Meg, A M. 1996 and Krieg, A M, et al., 1999).

Recent studies demonstrate that at least three classes of CpG DNA sequences exist, each with different physical characteristics and biological effects. Preliminary studies in several animal models of cancer suggest that CpG DNA may have many uses in cancer immunotherapy. CpG DNA have the ability to induce tumour regression by activating innate immunity, enhancing antibody dependent cellular cytotoxicity, and serving as potent vaccine adjuvants that elicit a specific, protective immune response. Early clinical trials indicate that CpG DNA can be administered safely to humans, and studies are ongoing to understand how these agents may play a role in cancer immunotherapy (Wooldridge, J E, et al., 2003)

An early patent (U.S. Pat. No. 6,498,147) presented antisense oligonucleotides and disclosed antisense inhibition of tumour cells in vitro, as well as an animal experiment showing antisense inhibition of tumour growth in vivo in syngenic C57B1/6 mice. The mice were treated with intraperitoneal injections of 40 mg/g sense and antisense oligodeoxynucleotides. Histologic analysis showed focal tumour necrosis followed by widespread segmental necrosis.

B-cell chronic lymphocytic leukemia (B-CLL) is the most common leukemia in the western world. B-CLL is a cancer of the white blood cells and bone marrow, characterized by uncontrolled proliferation and/or reduced cell death (apoptosis) of blood cells, specifically the B lymphocytes, and is the most widespread form of adult leukemia. Its incidence approaches 50 per 100,000 after the age of seventy. The leukemia usually has a protracted natural course of years and even decades, but eventually accelerates as the cells acquire sequential genetic defects. B-CLL differs from many other malignancies in that monoclonal B-CLL cells accumulate relentlessly, due to an abnormally prolonged life span, which likely is a consequence of altered interactions between defective B-CLL cells and their environment. Cytokines are essential factors in cell homeostasis and cell-cell dialogue, and are proposed to be critical in this milieu (Caligaris-Cappio et al., 1999 and Rozman et al., 1995).

No common initial transforming event has been found for B-CLL. Chromosomal translocations, thought to occur mainly during the gene rearrangement process and common in other lymphoid malignancies, are rare in B-CLL. Karyotypic abnormalities tend to increase in frequency and number during the course of the disease. When translocations are found, they tend to result in genetic loss rather than in the formation of a fusion gene or over-expression of an oncogene. The most common genetic abnormalities in B-CLL are 13q deletions (50% of cases), 13q4 deletions (associated with an indolent course), trisomy 12 (12g13-15, with over-expression of the MDMQ oncoprotein which suppresses p53, 25% of cases), 11q22-q23 deletions (loss of ATM, 10% of cases) and 17p deletions (deletion of p53) which causes resistance to apoptosis and the cancer often becomes refractory (Gaidan et al., 1991 and Dohner et al., 1999).

B-CLL cells express surface molecules such as CD23 (low affinity receptor for IgE), CD25 (IL-2R α chain), and CD27 (co-stimulatory molecule), which in other settings indicate a state of activation. The expression and association of several proteins tightly regulate the process of apoptosis. The relative balance of these proteins controls cell life span. Genes responsible for this system include the BCL-2 family, the tumour necrosis factor receptor and genes such as Myc and p53 (Osorio et al., 1999). All the death pathways promoted by these genes appear to have a common “demolition” cascade, represented by the protease family of the caspases. B-CLL cells consistently express high levels of products of the anti-apoptosis members of the BCL-2 family (bad-2, bcl-n, bax), while the Bcl-2 function inhibitor Bcl-6 is markedly reduced. The mechanism involved in overexpression of Bcl-2 is currently unclear. The leukemic cells of B-CLL are negative or weakly positive for Fas. They generally remain resistant to anti-Fas antibody mediated death even after stimulation induced Fas expression. In rare sensitive cases, cell death occurs independently of Bcl-2 expression by a mechanism still uncharacterized. It would appear that Bcl-2 overexpression and the Fas pathway are mechanisms involved in the pathophysiology of B-CLL but not necessarily critical causative events. Mediators including cytokines are likely to link the initial etiologic factor with the terminal pathways of apoptosis.

Most B-CLL cells are the in GO phase of the cell cycle and can not be induced to enter the proliferative phase by conventional methods such as concanavalin-A, phorbolesters, or receptor cross-linking, which induce the proliferation of normal lymphocytes. Only a small subset of cells appears to enlarge the clonal population in response to an unknown promoting signal. Proliferation promoting cytokines may provide this stimulus in vivo (Dancesco et al., 1992).

B-CLL cells accumulate at the expense of the normal B-cell pool. Total T-cells on the other hand, are usually increased. The bone marrow T-lymphocytes are predominantly CD4+ cells as seen in autoimmune disorders such as rheumatoid arthritis and sarcoidosis. There is frequently a Th2 predominant cytokine phenotype in peripheral blood. Abnormalities in the TCR repertoire have been reported also. Reports indicate that T-lymphocytes and stromal cells may have a key role in supporting an environment capable of perpetuating the life span of the B-CLL cells. Both the malignant cells and their T-cell entourage express a vanity of surface molecules and their receptors: CD5 and its ligand CD72, CD27 and CD70. These findings open various possibilities of mutual interaction which could result directly or indirectly (cytokines) in cell self-preservation. Such lengthy survival would, in turn increase chances for accumulation of gene mutations and genetic instability, which favours disease progression through dysregulation of cell cycle check-points, and resistance to cytotoxic therapy (Klein et al., 2000).

The symbiotic interaction between B-CLL cells and their environment is almost certainly mediated by the secretion of cytokines and modulated by adhesion molecules. Investigation of cytokine involvement in B-CLL has generated a substantial body of data supporting or disproving various cytokines as mediators of proliferation and prolonged life span in this leukemia. Cytokine production investigations have demonstrated reverse-transcription polymerase chain reaction signals for IL-1, IL-2, IL-3, IL-4, IL-5, IL-7, TNF-β, and TNF-α (Pistoia et al, 1997). These findings have been contradicted by other studies which showed negative results for IL-4, IL-3 and IL-6 (Tangye et al., 1999). In contrast, TGF-β as well as IL10 secretion, has been shown in normal B-lymphocytes. No other cytokine production has been reported to be constitutive for these cells.

Immunotherapy of cancer has been explored for over a century, but it is only in the last decade that various antibody-based products have been introduced into the management of patients with diverse forms of cancer. At present, this is one of the most active areas of clinical research, with eight therapeutic products already approved in oncology. Antibodies against tumour-associated markers have been a part of medical practice in immunohistology and in vitro immunoassays for several decades, and are now becoming increasingly recognized as important biological agents for the detection and treatment of cancer (Strome et al., 2007). Molecular engineering has improved the prospects for such antibody-based therapeutics, resulting in different constructs and humanized or human antibodies that can be frequently administered.

CD20 is variably expressed on the surface of B-cells in CLL patients with some patient's B-cells expressing very low levels of CD20 antigen. CD20 (human B-lymphocyte restricted differentiation antigen), is a hydrophobic transmembrane protein with a molecular weight of approximately 35 kD located on pre-B and mature B lymphocytes. The antigen is also expressed on more than 90% of B-cells in non Hodgkin's lymphomas (NHL), but is not found on hematopoietic stem cells, pro B cells, normal plasma cells or other normal tissues. CD20 regulates an early step(s) in the activation process for cell cycle initiation and differentiation, and possibly functions as a calcium ion channel. CD20 is not shed from the cell surface and does not internalize upon antibody binding. Free CD20 antigen is not found in the circulation (Pescovitz, 2006).

The anti-CD20 antibody rituximab, which is a genetically engineered chimeric murine/human monoclonal antibody directed against human CD20 (Rituxan® or MabThera®, from Genentech, Inc., South San Francisco, Calif., U.S.) is used for the treatment of patients with relapsed or refractory low-grade or follicular, CD20 positive, B-cell non-Hodgkin's lymphoma and B-CLL. Rituximab works by recruiting the body's natural defences to attack and kill the B-cell to which it binds via the CD20 antigen. In vitro mechanism of action studies have demonstrated that rituximab binds human complement and lyses lymphoid B-cell lines through complement-dependent cytotoxicity (CDC) (Reff et al., 1994). Additionally, it has significant activity in assays for antibody-dependent cell-mediated cytotoxicity (ADCC). In vivo preclinical studies have shown that rituximab depletes B-cells from the peripheral blood, lymph nodes, and bone marrow of cynomolgus monkeys, presumably through complement and cell-mediated processes (Reff et al., 1994). While rituximab has been used with some success in CLL patients, analysis of CLL patients shows that the density of CD20 on the surface of B-CLL cells is rather variable with some patient's B cells expressing very low levels of the CD20 antigen. Furthermore, a recent clinical trial where rituximab was administered in combination with PF-3512676 (formerly CpG 7909, a TLR9 activating oligonucleotide) to treat lymphoma, failed to show the desired results (Leonard et at, 2007).

The typical treatment for B-cell malignancies, besides rituximab, is the administration of radiation therapy and chemotherapeutic agents. In the case of CLL, conventional external radiation therapy will be used to destroy malignant cells. However, side effects are a limiting factor in this treatment. Another widely used treatment for haematological malignancies is chemotherapy. Combination chemotherapy has some success in reaching partial or complete remissions. Unfortunately, these remissions obtained through chemotherapy are often not durable.

Conversely, CD23 expression has been found to be consistently present at higher levels in B-CLL. The CD23 leukocyte differentiation antigen is a 45 kD type II transmembrane glycoprotein expressed on several haematopoietic lineage cells, which function as a low affinity receptor for IgE (FcγRII) (Pathan et al., 2008). It is a member of the C-type lectin family and contains an α-helical coiled-coil stalk between the extracellular lectin binding domain and the transmembrane region. The stalk structure is believed to contribute to the oligomerization of membrane-bound CD23 to a trimer during binding to its ligand (for example, IgE). Upon proteolysis, the membrane bound CD23 gives rise to several soluble CD23 (sCD23) molecular weight species (37 kD, 29 kD and 16kD). In addition to being involved in regulating the production of IgE, CD23 has also been speculated to promote survival of germinal center B cells. The expression of CD23 is highly up-regulated in normal activated follicular B cells and in B-CLL cells.

Lumiliximab is a monoclonal chimeric anti-CD23 antibody (from Biogen Idec, currently undergoing clinical trials) that harbours macaque variable regions and human constant regions (IgG1, κ) and was originally developed to inhibit the production of IgE by activated human blood B-cells. It is now in a Phase III trial for use in B-CLL patients. In vitro studies have shown that lumiliximab induces caspase dependent apoptosis in B-CLL cells through the mitochondrial death pathway (Pathan et al., 2008). Thus, it seems to induce apoptosis of tumour cells through a mechanism different from rituximab.

Several other antibodies have recently been approved for the treatment of cancer. Alemtuzumab (Campath® or MabCampath®, an anti-CD52 from Ilex Pharmaceuticals) (Keating et al., 2002) was approved in 2001 for the treatment of refractory CLL. Bevacizumab (Avastin®, Genentech, Inc., South San Francisco, Calif.) is a humanized IgG1 mAb directed against vascular endothelial growth factor (VEGF) used in treatment of colorectal cancer, small cell lung cancer and breast cancer. Trastuzumab (Herceptin® from Roche) is a humanized IgG1 mAb that is effective against metastatic breast cancer tumours over-expressing the HER-2 target (Strome et al., 2007).

Ofatumumab (HuMax-CD20, GlaxoSmithKline) and Veltuzumab (Immunomedics) have also been proposed for the treatment of cancer (e.g. CLL).

In order to make antibody drugs more efficient, an up-regulation of the specific antigen targets on the surface of tumour cells might be helpful. One way of obtaining such an effect could be to stimulate the cells with immunomodulatory oligonucleotides. Immune stimulatory effects can be obtained through the use of synthetic DNA-based oligodeoxynucleotides (ODN) containing unmethylated CpG motifs. Such CpG ODN have highly immunostimulatory effects on human and murine leukocytes, inducing B cell proliferation; cytokine and immunoglobulin secretion; natural killer (NK) cell lytic activity and IFN-gamma secretion. CpG ODN also activate dendritic cells (DCs) and other antigen presenting cells, leading to expression of co-stimulatory molecules and secreted cytokines, especially the Th1-like cytokines that are important in promoting the development of Th1-like T cell responses (Krieg et al, 1995). The increase in receptor density by CpG-ODNs could be mediated through a direct effect of the oligonucleotides on the cells, or through the induction of cytokines. An increase in antigen density or an increase in the population of cells expressing the target receptors would enable the antibodies to kill the tumour cells more efficiently, either through enhancing antibody-dependent cell-mediated cytotoxicity (ADCC) or complement-dependent cytotoxicity (CDC).

There are indications that the CpG motif alone is not accountable for the efficacy of the oligonucleotides. There are even indications that this motif is not necessary for the desired function.

Regardless of the considerable effort spent on developing oligonucleotide based therapeutic approaches to cancer, and the occasional success reported so far, there still remains a need for new compounds and modes of administration, exhibiting improved efficacy and minimal or no side effects.

Antibody therapy in general is costly, and there is a need for improvements inter alia with regards to efficacy.

### SUMMARY

The present inventors have surprisingly found that specific oligonucleotide sequences when given subcutaneously or in particular when administered topically on a mucous membrane, e.g. orally, pulmonary, intranasally, rectally, or intravaginally, have a profound effect on various human cancer forms as confirmed in vivo, in animal studies, and in vitro, using PBMCs from CLL patients and healthy subjects.

Further, novel sequences have been developed and tested in animal experiments in vivo and in human material in vitro, showing pronounced therapeutic effects either alone or in combination with other treatments. The oligonucleotides are used to induce apoptosis, and in particular to increase the expression of cell surface receptors. The inventive oligonucleotides can be used in combination with immunological approaches to treat cancer, in particular monoclonal antibodies directed to specific receptors. Embodiments of the invention are defined in the attached claims, incorporated herein by reference.

### BRIEF DESCRIPTION OF THE DRAWINGS

The invention will be described in closer detail in the following description, non-limiting examples and claims, with reference to the attached drawings in which

FIG. 1. (A) is a graph showing tumour growth measured as tumour volume (mm³) over time for mice with induced subcutaneous RMA lymphoma, following subcutaneous administration of 50 μg of the substances of SEQ ID NO. 1 and 2, compared to control (PBS). (B) is a graph showing tumour growth measured as tumour volume (mm³) over time for mice with induced subcutaneous RMA lymphoma, following subcutaneous administration of 50 or 150 μg, or intranasal administration of 50 μg of the substance of SEQ ID NO. 4.

FIG. 2A is a bar diagram showing the growth reducing effect on the human colon cancer cell line HCT116 in vitro, following 72 hrs of treatment with the compounds according to SEQ ID NO. 1-4, wherein “−” denotes a negative control. Cell growth was measured by flow cytometry of Ki-67 positive cells. Bars represent the relative growth of treated cells compared to untreated (M) cells±SEM.

FIG. 2B is a bar diagram showing induction of apoptosis in the human colon cancer cell line HCT116 in vitro, following 72 hrs of treatment with the compounds according to SEQ ID NO. 1-4, wherein “−” denotes a negative control. Apoptosis was measured by flow cytometry of 7-AAD positive cells.

FIG. 2C consists of a bar diagram showing the surface expression of the B-cell proliferation marker CD20 in a human B-cell lymphoma cell line in vitro, following 48 hrs of treatment with compounds according to SEQ ID NO. 1, 3 and 4. Surface expression of CD20 was measured by flow cytometry. “−” denotes a negative control. Bars represent the relative mean fluorescent intensities (MFI) of treated cells compared to untreated (M) cells.

FIG. 2D is a graph showing cell survival of the human Burkitt's lymphoma cell line in vitro, following 72 hrs of treatment with the compounds according to SEQ ID NO. 1, 3 and 4, wherein “−” denotes a negative control. Cell survival was measured by counting cells daily for 3 days after start of treatment, excluding Trypan blue positive cells. Lines represent the relative cell survival of treated cells compared to untreated (M) cells.

FIG. 3 is a graph showing how 48 hrs of treatment with the experimental compounds induce up-regulation of CD20 (FIG. 3A), CD23 (FIG. 3B) and CD80 (FIG. 3C) on CD19 positive B-cells from CLL-patients as measured by flow cytometry. All compounds (SEQ ID NO. 1-8) were tested at the concentrations, 1, 10 and 25 μM. Bars represent the mean MFI values±SEM of the CD20 surface expression in 18 samples. “−” denotes a negative control.

FIG. 3D shows how 48 hrs of treatment with the experimental compounds induce activation of NK-cells in PBMCs from CLL-patients as measured by staining CD69 positive/CD56 positive cells using flow cytometry. The compounds are represented by SEQ ID NO. 1-7. “−” denotes a negative control. Bars represent the mean percentages±SEM of activated NK-cells in 18 samples.

FIG. 3E shows that treatment with the experimental compounds for 72 hrs induce apoptosis of B-cells in PBMCs from CLL-patients. All compounds (SEQ ID NO. 1-6) were tested at the concentrations 1, 10 and 25 μM. Apoptosis was measured by 7-AAD staining of CD19 positive cells and subsequently analyzed by flow cytometry. Bars represent the mean percentages±SEM of induced apoptosis in 10 samples.

FIG. 4A shows the increased production of the cytokine IL-6 in healthy PBMCs treated with SEQ ID NO. 1 at the concentration of 25 μM following 30 min, 2 hrs and 6 hrs exposure to the compound, compared to untreated cells.

FIG. 4B shows the increased production of the cytokine IL-10 in healthy PBMCs treated with SEQ ID NO. 1 at the concentration of 25 μM following 30 min, 2 hrs and 6 hrs exposure to the compound, compared to untreated cells.

FIG. 4C shows the increased production of the cytokine IP-10 in healthy PBMCs treated with SEQ ID NO. 1 at the concentration of 25 μM following 30 min, 2 hrs and 6 hrs exposure to the compound, compared to untreated cells.

FIG. 4D shows the up-regulation of CD20 surface expression on CLL B cells treated with SEQ ID NO. 1 at the concentrations 0.1, 1, 10 and 25 μM following 2 hrs, 6 hrs and 24 hrs exposures to the compound, compared to cells treated continuously for 72 hrs and untreated cells. CD20 expression was analyzed by flow cytometry and bars represent the mean percentages±SEM of CD20 surface expression from 4 patient samples.

FIG. 4E shows the activation of NK-cells in CLL-PBMCs treated with SEQ ID NO. 1 at the concentrations 0.1, 1, 10 and 25 μM following 2 hrs, 6 hrs and 24 hrs exposures to the compound, compared to cells treated continuously for 72 hrs and untreated cells. Activation of NK cells was analyzed by FACS measuring the percentage of CD69 positive CD56 positive cells. Bars represent the mean percentages±SEM from 4 patient samples.

FIG. 5A-E illustrates the enhanced efficacy of rituximab in vitro on B cells from human CLL patients. CLL B cells were pre-treated with inventive compounds; SEQ ID NO. 1 (FIG. 5A), SEQ ID NO. 3 (FIG. 5B), SEQ ID NO. 4 (FIG. 5C), SEQ ID NO. 7 (FIG. 5D) or SEQ ID NO. 8 (FIG. 5E) for 48 hrs, and subsequently treated with rituximab for 24 hrs for analysis of apoptosis mediated through ADCC (FIG. 5A-E). Bars represent the mean percentages±SEM of apoptosis of CD19 positive CLL cells as measured by double staining of CD19 positive cells with Annexin V and 7-AAD. n=18.

FIG. 5F shows cell death mediated through CDC. CLL B cells were pre-treated with inventive compounds; SEQ ID NO. 1 (FIG. 5F), SEQ ID NO. 3, 4, 7 or 8 (data not shown) for 48 hrs, and subsequently treated with rituximab in medium supplemented with 30% human serum for 4 hrs for analysis of apoptosis mediated through CDC (FIG. 5F). Bars represent the mean percentages±SEM of apoptosis of CD19 positive CLL cells as measured by double staining of CD19 positive cells with Annexin V and 7-AAD. n=18.

FIG. 5G illustrates the importance of the order of administration, wherein FIG. 5A shows the mean percentages±SEM of apoptosis when the expression of CD20 was increased by SEQ ID NO. 1 before the administration of rituximab, and FIG. 5G shows the corresponding results when rituximab was added 48 hrs prior to SEQ ID NO. 1. n=10.

FIG. 6 shows the induction of cytokines in CLL-samples responding well to combination treatment versus samples responding weakly to combination treatment. Cell supernatants were harvested after 48 hrs of treatment with SEQ ID NO. 1-6 and subsequently analyzed by cytometric bead array (CBA) for the content of IL-6 (FIG. 6A), IL-10 (FIG. 6B), IL-12 (FIG. 6C), IP-10 (FIG. 6D) and TNF-α (FIG. 6E).

### DESCRIPTION

Before the invention is described in detail, it is to be understood that this invention is not limited to the particular sequences described or steps of the methods described as such sequences and methods may vary. It is also to be understood that the terminology used herein is for purposes of describing particular embodiments only, and is not intended to be limiting. It must be noted that, as used in the specification and the appended claims, the singular forms “a,” “an” and “the” also include plural referents unless the context clearly dictates otherwise. Thus, for example, reference to “a sequence” includes more than one such sequence, and the like.

Further, the term “about” is used to indicate a deviation of +/−2% of the given value, preferably +/−5% and most preferably +/−10% of the numeric values, when applicable.

The term “cancer” is meant to mean any malignant neoplastic disease, i.e. any malignant growth or tumour caused by abnormal and uncontrolled cell division. The term “cancer” is in particular meant to include both solid, localized tumours, as exemplified in the animal experiments included in the present description, and non-solid cancer forms, such as but not limited to chronic lymphocytic leukaemia (CLL), one form of leukaemia investigated in the examples.

The term “immunomodulatory” refers to an immune response either stimulating the immune system or repressing the immune system or both in an organism when administered to a vertebrate, such as a mammal. As used herein, the term “mammal” includes, without limitation rats, mice, cats, dogs, horses, cattle, cows, pigs, rabbits, non-human primates, and humans.

The term “immunomodulatory response” describes the change of an immune response when challenged with an immunomodulatory oligonucleotide. This change is measurable often through the release of certain cytokines such as interferons as well as other physiological parameters such as proliferation. The response can equally be one that serves to stimulate the immune system as well as to repress the immune system depending on the cytokines induced by the immunomodulatory oligonucleotide in question.

The experiments performed using human cell lines in vitro indicate that the oligonucleotides according to the invention are capable of both reducing growth and inducing apoptosis. In addition, a reduction in dose in vivo (from 150 μg to 50 μg) significantly improved the response in subcutaneous administration. Surprisingly, application on a mucous membrane, here tested in the form of nasal administration, provided an equally effective way of administration in a mouse model.

The inventors also found that the inventive compounds are capable of eliciting or increasing the expression of cell surface markers, here illustrated by the cell surface markers CD20, CD23, CD69 and CD80.

The inventors therefore make available, as one embodiment of the invention, compounds and methods for the treatment of cancer, wherein the inventive compounds presented in Table 1 are used either alone; to increase apoptosis, and/or to up-regulate the expression of one or more of the cell surface markers CD20, CD23, CD69 and CD80; or in combination with an anti-tumour therapy chosen among radiation treatment, hormone treatment, surgical removal of the tumour, chemotherapy, immunological or immunomodulatory therapies, photodynamic therapy, laser therapy, hyperthermia, cryotherapy, angiogenesis inhibition, or a combination of any of these. Most preferably said anti-tumour treatment is an immunological or immunomodulatory treatment and comprises the administration of an antibody to the patient.

Examples of presently available antibodies include, but are not limited to, rituximab (Rituxan®, MabThera®), alemtuzumab (Campath®, MabCampath®), bevacizumab (Avastin®), and trastuzumab (Herceptin®).

When given in combination with an anti-tumour therapy, the inventive compounds are preferably administered in advance of the anti-tumour therapy, preferably at least about 12 hours, more preferably about 24 hours, and most preferably about 48 hours in advance of the therapy. When given in combination with an immunological therapy, and in particular a therapy involving the administration of an antibody, the inventive compound is preferably administered before the administration of the antibody to the patient, and most preferably sufficiently before in order to allow for the up-regulation of a cell surface molecule or cell surface marker towards which the specific antibody is targeted.

The invention makes available specific nucleotides, i.e. the isolated oligonucleotide sequences according to any one of SEQ ID NO. 1-7. See Table 1.

TABLE 1
Sequence information
Table 1

| SEQ | | |
|---|---|---|
| ID | | |
| NO. | Sequence (5′-3′) | IDX-No |
| 1 | T*C*G*TCGTTCTGCCATCGTC*G*T*T | 9022 |
| 2 | G*G*G*GTCGTCTG*C*G*G | 9052 |
| 3 | G*A*T*CGTCCGTCGG*G*G*G | 9058 |
| 4 | G*G*A*ACAGTTCGTCCAT*G*G*C | 0150 |
| 5 | T*C*G*TCGTTCGGCCGATCG*T*C*C | 9038 |
| 6 | T*C*G*TTCGTCTGCTTGTTC*G*T*C | 9071 |
| 7 | G*G*A*A*C*A*G*T*T*G*C*T*C*C*A*T*G*G*C | 0505 |
| 8 | C*C*G*GGGTCGCAGCTGAGCCCA*C*G*G | 0011 |

Note: * denotes phosphothioation

The above sequences SEQ ID NO. 1-7 have been designed by the inventors, and are with the exception of SEQ ID NO. 4, to the best knowledge of the inventors, not previously known. SEQ ID NO. 4 was published for the first time in 1993 (Sokoloski et al. 1993).

SEQ NO 7 is a fully phosphorothioated IDX0150 (SEQ ID NO. 4), containing a GC instead of a CG, i.e. without an CpG-motif.

SEQ ID NO. 8 is used as a negative control only and is not included in the claims.

The oligonucleotide sequence according to any one of SEQ ID NO. 1-7 may comprise at least one nucleotide having a phosphate backbone modification. Said phosphate backbone modification is preferably a phosphorothioate or phosphorodithioate modification.

The present invention also comprises the use of an isolated oligonucleotide sequence according to any one of SEQ ID NO. 1-3 and 5-7 for the manufacture of a medicament for the treatment of cancer.

In particular, the use of an isolated oligonucleotide sequence according to any one of SEQ NO 1-7 for the manufacture of a medicament for the treatment of cancer through induction of apoptosis and/or increased expression of a cell surface marker.

Correspondingly, the invention also comprises the use of an isolated oligonucleotide sequence according to any one of SEQ ID NO. 1-7 for the manufacture of a medicament for subcutaneous administration in a dose effective to achieve at least one of up-regulation of a cell surface marker and/or induction of apoptosis in the treatment of cancer. Said dose is preferably in the interval of about 0.01 to about 50 mg/kg, more preferably 0.05 to about 5 mg/kg and most preferably 0.1 to about 1 mg/kg for the treatment of cancer.

In particular sequences SEQ ID NO. 1, 4 and 6 are shown to be promising up-regulators of cell surface markers, in particular CD20, as shown in CLL B cells.

The medicament can be administered subcutaneously, nasally, orally, intravenously, or mucosally, e.g. orally, topically to a mucous membrane, rectally, vaginally, by inhalation etc.

A preferred embodiment of the invention comprises the use as defined above, wherein an anti-tumour treatment is administered before, after or essentially simultaneously with the administration of said oligonucleotide. This anti-tumour treatment is chosen among radiation treatment, hormone treatment, surgical removal of the tumour, chemotherapy, immunological or immunomodulating therapy, photodynamic therapy, laser therapy, hyperthermia, cryotherapy, angiogenesis inhibition, or a combination of any of these.

The anti-tumour treatment is preferably an immunological or immunomodulating therapy, such as a therapy involving the administration of an antibody to the patient. In the case of an immunological treatment, such as the administration of an antibody, the inventive compound is preferably administered before the administration of the antibody. The time period is chosen so that the desired up-regulation of expression of cell surface markers is achieved, and is preferably at least about 12 hours, more preferably about 24 hours, and most preferably about 48 hours prior to administration of the antibody. It is also conceived that an additional dose of the inventive compounds may have to be given after the administration of the antibody, to boost the up-regulation of the cell surface markers.

The use of the above described anti-tumour treatment, wherein the oligonucleotide sequence according to any one of SEQ ID NO. 1-7 may comprise at least one nucleotide having a phosphate backbone modification. Said phosphate backbone modification is preferably a phosphorothioate or phosphorodithioate modification.

Consequently the present invention also comprises a method for the treatment of cancer wherein an isolated oligonucleotide sequence according to any one of SEQ ID NO. 1-3 and 5-7 is administered to a patient in need thereof.

As defined above, at least one nucleotide in any one of SEQ ID NO. 1-3 and 5-7 may contain a phosphate backbone modification. Said phosphate backbone modification is preferably a phosphorothioate or phosphorodithioate modification.

According to an embodiment of the method of treatment according to the invention, said oligonucleotide is administered mucosally, i.e. topically to a mucous membrane of a patient in need thereof. Mucosal administration includes oral, pulmonary, rectal, vaginal, and nasal administration. Preferably, said oligonucleotide is administered in a dose of about 0.01 to about 50 mg/kg, more preferably 0.05 to about 5 mg/kg and most preferably 0.1 to about 1 mg/kg body weight.

According to another embodiment, the oligonucleotide is administered subcutaneously to a patient in need thereof. Preferably, said oligonucleotide is administered in a dose of about 0.01 to about 50 mg/kg, more preferably 0.05 to about 5 mg/kg and most preferably 0.1 to about 1 mg/kg.

The present inventors have confirmed in human material in vitro that the oligonucleotides according to SEQ ID NO. 1, 3, 4 and 7 exert a synergistic effect when used in combination with other approaches to the treatment of cancer. Thus, according to an embodiment of the invention, said oligonucleotide is administered before or essentially simultaneously with an anti-tumour treatment, most preferably before an anti-tumour treatment, in particular when said anti-tumour treatment involves the administration of an antibody.

As outlined above, this anti-tumour treatment is chosen among radiation treatment, hormone treatment, surgical removal of the tumour, chemotherapy, immunological or immunomodulating therapy, photodynamic therapy, laser therapy, hyperthermia, cryotherapy, angiogenesis inhibition, or a combination of any of these.

The anti-tumour treatment is preferably an immunological therapy involving the administration of an antibody to the patient. Examples of antibodies include antibodies currently in use as well as under evaluation, e.g. rituximab, ocrelizumab, altuzumab, ofatumumab, tositumomab, ibritumomab (directed to CD20), lumiliximab (CD23), alemtuzumab (CD52), galiximab (CD80), epratuzimab (CD22), and daclizumab (CD25).

In one embodiment the anti-tumour treatment of cancer, wherein an isolated oligonucleotide sequence according to any one of SEQ ID NO. 1-3 and 5-7 is administered to a patient in need thereof. Said oligonucleotide is administered topically to a mucous membrane or subcutaneously to a patient in need thereof.

In another embodiment of the treatment of cancer, an oligonucleotide sequence chosen among SE ID NO 1-7, is administered in a dose effective to elicit the expression of at least one of the cell surface markers CD20, CD23, CD69 and CD80. Said at least one oligonucleotide has a phosphate backbone modification and is administered in a dose of about 0.01 to about 50 mg/kg body weight, more preferably 0.05 to about 5 mg/kg body weight and most preferably 0.1 to about 1 mg/kg body weight. Said oligonucleotide may be is administered before or essentially simultaneously with an anti-tumour treatment, wherein the anti-tumour treatment is chosen among radiation treatment, hormone treatment, surgical removal of the tumour, chemotherapy, immunological or immunomodulating therapy, photodynamic therapy, laser therapy, hyperthermia, cryotherapy, angiogenesis inhibition, or a combination of any of these. Said anti-tumour treatment is an immunological treatment and comprises the administration of said oligonucleotide sequence before or in combination of an antibody to the patient.

In any one of the above embodiments of the invention, said oligonucleotide is administered in a dose effective to elicit or increase or up-regulate the expression of at least one cell surface molecule or cell surface marker, in particular a cell surface marker chosen among CD20, CD23, CD69 and CD80. Said oligonucleotide may have a phosphate backbone modification.

A skilled person is well aware of the fact that there are numerous approaches to the treatment of cancer. It is characteristic for the battle against cancer that several therapies are used, depending on the type of cancer, its location and state of progression, and the condition of the patient. It is frequently so that several therapies are used subsequently, or in combination. While some therapies such as surgical intervention, radiation therapy and chemotherapy have been practiced for many decades, others have been recently conceived and many are still in experimental use. Naturally new approaches are constantly being developed, and it is conceived that the oligonucleotides, their use and methods of treatment according to the present invention, will find utility also in combination with future treatments. The inventors presently believe that the inventive oligonucleotides, their use and methods of treatment would be useful in combination with the following anti-tumour treatments, however without wishing to be limited to the same; radiation treatment, hormone treatment, surgical intervention, chemotherapy, immunological or immunomodulating therapy, photodynamic therapy, laser therapy, hyperthermia, cryotherapy, angiogenesis inhibition, or a combination of any of these.

The anti-tumour treatment is preferably an immunological or immunomodulating therapy involving the administration of an antibody to the patient.

The oligonucleotide is administered in a therapeutically effective dose. The definition of a “therapeutically effective dose” is dependent on the disease and treatment setting, a “therapeutically effective dose” being a dose which alone or in combination with other treatments results in a measurable improvement of the patient's condition.

According to an embodiment, the oligonucleotide is administered subcutaneously in an amount of about 0.01 to about 50 mg per kg body weight. Preferably the oligonucleotide is administered in an amount of about 0.05 to 5 mg per kg body weight. Most preferably the oligonucleotide is administred in an amount of about 0.1 to 1 mg per kg body weight.

The oligonucleotide may be administered in a single dose or in repeated doses administered subcutaneously, intravenously, or to a mucous membrane, e.g. given orally, intranasally, rectally or intravaginally.

The nucleotides according to the invention can be delivered subcutaneously or topically on a mucous membrane. The term “topically on a mucous membrane” includes oral, pulmonary, rectal, vaginal, and nasal administration. The nucleotides can be delivered in any suitable formulation, such as suitable aqueous buffers, for example but not limited to phosphate buffered saline (PBS). It is contemplated that the nucleotides are administered in a suitable formulation, designed to increase adhesion to the mucous membrane, such as suitable gel-forming polymers, e.g. chitosan etc; a formulation enhancing the cell uptake of the nucleotides, such as a lipophilic delivery vehicle, liposomes or micelles; or both. There are several methods and devices available for nasal administration; single or multi-dosing of liquid formulations, powder formulations and spray formulations with either topical or systemic action. The present invention is not limited to particular methods or devices for administering the nucleotides to the nasal mucous membrane. The initial animal studies have shown that simple instillation by pipette works satisfactorily, although for human use, devices for reliable single or multi dose administration would be preferred.

Preferably, the route of administration of said medicament is chosen from, subcutaneous, intravenous, intramuscular, mucosal and intraperitoneal administration. Preferably the mucosal administration is chosen from oral, gastric, nasal, ocular, rectal, urogenital and vaginal administration.

According an embodiment, the oligonucleotide is administered by intravenous injection or infusion.

According to another embodiment the oligonucleotide is administered subcutaneously to a patient in need thereof.

The inventors also make available pharmaceutical compositions comprising an oligonucleotide according to any one of SEQ ID NO. 1-3 and 5-7. Said pharmaceutical compositions further preferably comprise a pharmacologically compatible and physiologically acceptable excipient or carrier, chosen from saline, liposomes, surfactants, mucoadhesive compounds, enzyme inhibitors, bile salts, absorption enhancers, cyclodextrins, or a combination thereof.

According to another embodiment of the invention, the oligonucleotides are administered to the mucous membrane of the colon through rectal instillation, e.g. in the form of an aqueous enema comprising the oligonucleotides suspended in a suitable buffer.

According to another embodiment of the invention, the oligonucleotides are administered to the mucous membrane of the lungs or the airways through inhalation of an aerosol, comprising the oligonucleotides suspended in a suitable buffer, or by performing a lavage, also comprising the oligonucleotides suspended in a suitable buffer.

According to yet another embodiment of the invention, the oligonucleotides are administered to the mucous membrane of the urogenital tract, such as the urethra, the vagina etc through application of a solution, a buffer, a gel, salve, paste or the like, comprising the oligonucleotides suspended in a suitable vehicle.

Although the effect from application to the nasal mucosa has been shown to be systemic, it is contemplated that application to other locations, such as the mucous membranes of the urogenital tract, the airways or the intestines, is more suitable for the treatment of tumours located in these organs or in the vicinity thereof.

The invention finds utility in the treatment of cancer, as supported by the in vivo and in vitro data presented in the experimental section and illustrated in the attached figures.

The embodiments of the invention have many advantages. So far, the administration of an oligonucleotide in the doses defined by the inventors has not elicited any noticeable side-effects. Further, the mucosal administration is easy, fast, and painless, and surprisingly results in a systemic effect. The influence on the conditions at the site of the tumour is believed to be one, but not the only, factor responsible for the reduction of growth and induction of apoptosis seen in the experiments. It is held that this effect, either alone, or in combination with existing and future anti-cancer treatments, offers a promising approach to battling cancer.

### EXAMPLES

### 1. Animal Experiments

The effect of subcutaneous growth of RMA lymphoma cells was investigated in vivo, in syngeneic C57BL/6 (B6) mice following administration of oligonucleotides. The objective of the study was to investigate the tumour growth inhibitory effect of different oligonucleotides in an experimental murine model of subcutaneous tumour growth. It is known that experimental subcutaneous tumours can be induced by inoculation of recipient B6 mice with in vivo maintained RMA tumour cells.

### 1.1 Test Systems

### Tumour Cell Type and Induction

Induction of a subcutaneous tumour in mice was achieved by inoculation of a cell suspension (10³) of in vivo-grown Raucher virus-induced lymphoma cells (RMA) into the right flank of the animal.

### Test Article Formulation and Preparation

SEQ ID NO. 1, 2 and 4 were supplied and delivered by Index Pharmaceuticals AB, Stockholm, Sweden, in “ready to use” concentrations (2.5-1.25 μg/μL) and kept at 4° C. until use.

### 1.2 Animal Material and Conditions

### Species, Strain and Supplier

The mice used were inbred C57BL/6/By mice obtained through in house breeding at MTC, Karolinska Institutet, Stockholm, Sweden.

### 1.3 Experimental Procedures/Experimental Design

### Experimental Procedures

In brief, the experiment comprised the following actions: RMA tumour cells were grown as an ascites tumour in B6 mice to provide a source of tumour cells adapted to in vivo growth. After retrieval, a low dose of RMA tumour cells (10³ cells) was inoculated into the right flank in recipient B6/By mice.

After tumour cell inoculation, all mice were monitored twice per week by palpation at the site of injection. At the first signs of tumour growth in any mouse, the mice were subdivided into groups and given 3 doses (100 μl) at one dose of the test substances every three days. The test substances were given subcutaneously in the left flank of the animals. In one group of mice, 50 μg (40 μl) of SEQ ID NO. 4 was administered intranasally. One group of control animals received 100 μl injections of the vehicle only (PBS).

### Evaluation of Tumour Growth Rate

The mice were continuously monitored and each mouse was followed by manual palpation. As soon as a tumour appeared, the growths of the subcutaneous tumours were measured daily using a caliper and expressed as cancer mass volume (mm³).

### Terminal Procedures

The tumour-bearing animals were sacrificed when the size of its growing tumour reached 1500 mm³. Any animal not developing a tumour was monitored for a maximum of two months, at which point the mouse was sacrificed.

### 1.4 Results

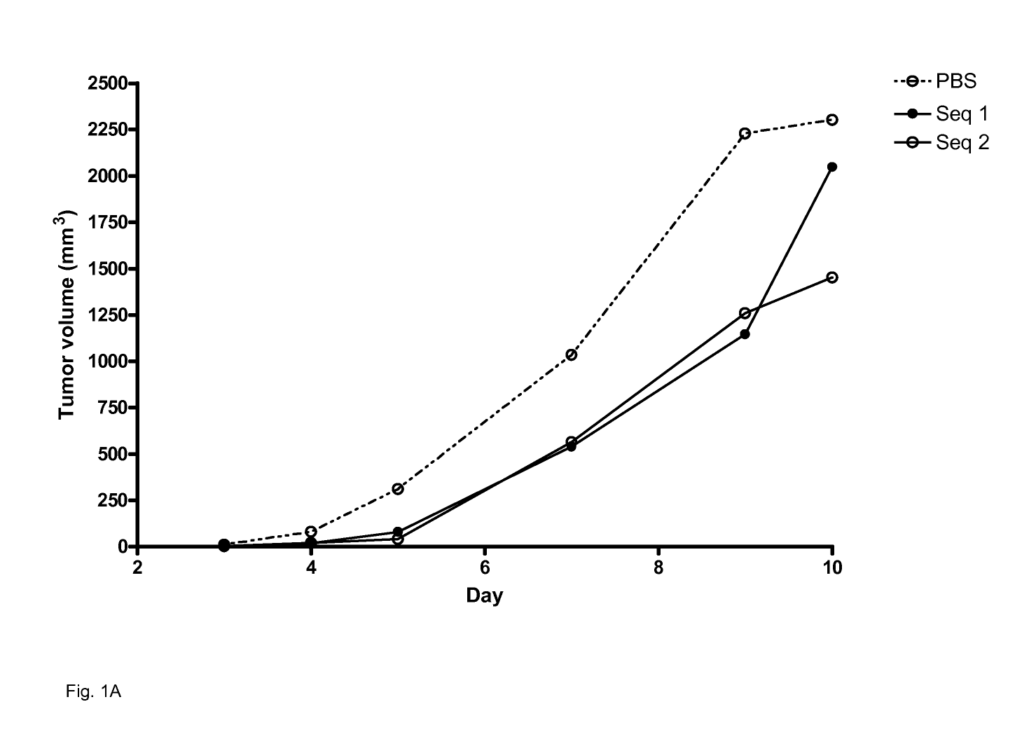

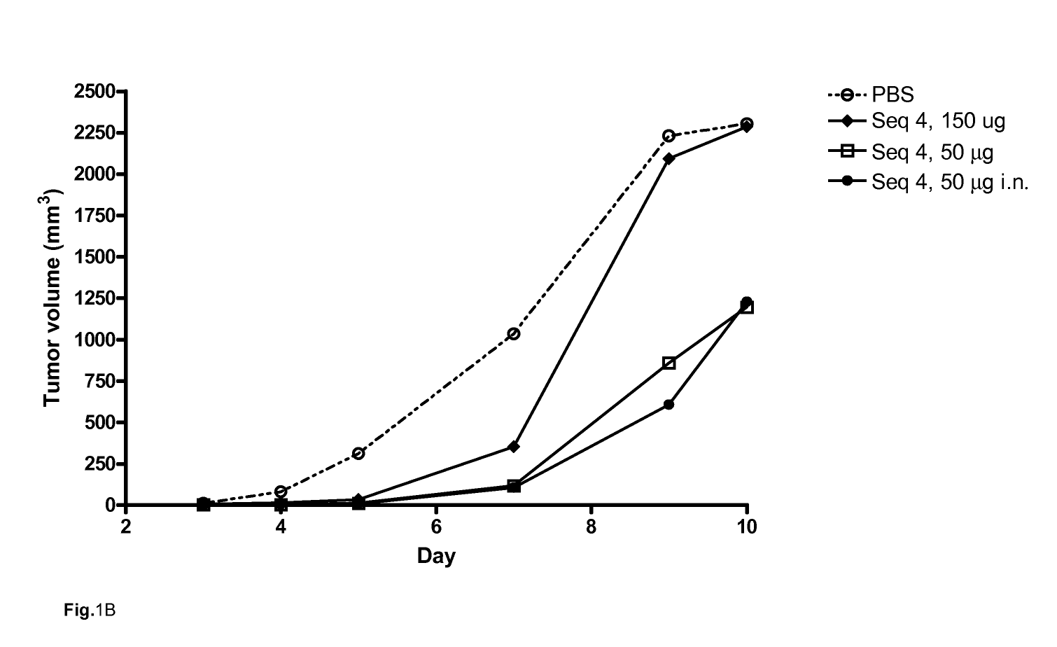

Each tested compound showed an inhibitory effect on tumour growth during the observation period of a maximum of 10 days (FIGS. 1A and 1B). SEQ ID NO. 1 and 2 showed equal abilities to reduce tumour growth in this experimental setting (FIG. 1A).

SEQ ID NO. 4 also reduced tumour growth (FIG. 1B). Surprisingly, a lower dose (50 μg vs. 150 μg) resulted in a pronounced reduction of tumour growth. Equally surprisingly, the same dose (50 μg) when administered intranasally resulted in an equally large tumour growth reduction (See FIG. 1B).

2. In Vitro Experiments with Human Cell Lines

Two recognized human tumour model cell lines were used. The objective of the study was to investigate the capability of different oligonucleotides to inhibit tumour cell growth and to induce apoptosis of tumour cells. A second objective was to study the effects obtained in animal studies in another set-up, predictive for the effect on cancer in humans. A negative control lacking a CpG motif was used.

### 2.1 Human Colon Cancer Cell Line

The human colon cancer cell line HCT116 was treated with each of the inventive nucleotides, SEQ ID NO. 1-4 in tissue culture medium for 72 hrs. Cell proliferation and cell death was analyzed by FACS analysis using Ki-67 and 7-amino actinomycin (7-AAD) staining, respectively, according to procedures known to a skilled person. Ki-67 is expressed by proliferating cells, and using 7-AAD, apoptotic cells could be identified.

### 2.2 Human Lymphoma Cell Line

The human Burkitt's lymphoma cell line Daudi was stimulated with each of the inventive nucleotides, SEQ ID NO. 1, 3 and 4 in tissue culture medium for 24, 48 and 72 hrs. The expression of various surface expression markers was analyzed by FACS (BD Biosciences, San Jose, Calif., USA) as described in literature (see e.g. Gursel, et al., 2002; Jahrsdorrer, et al., 2001; Jahrsdorler, et al., 2005a; Jahrsdorfer, et al., 2005b).

### 2.3 Results

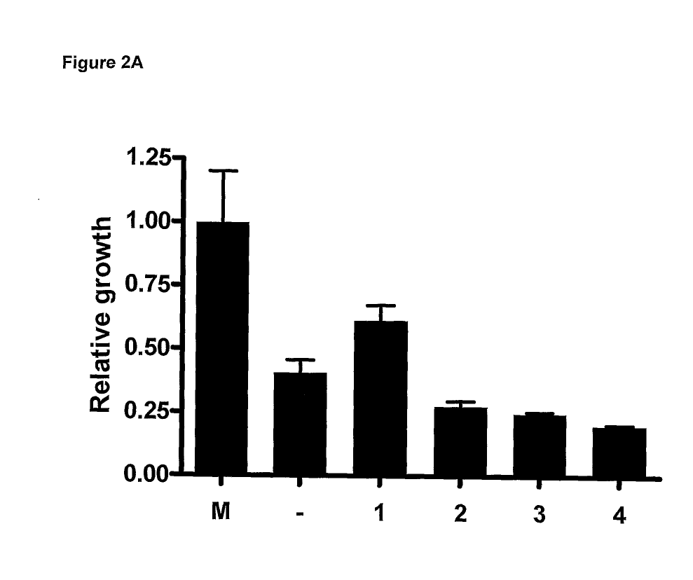

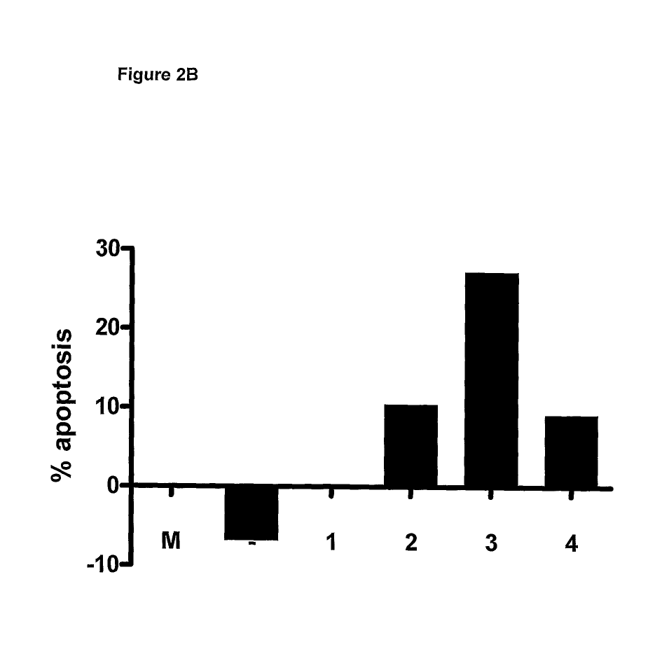

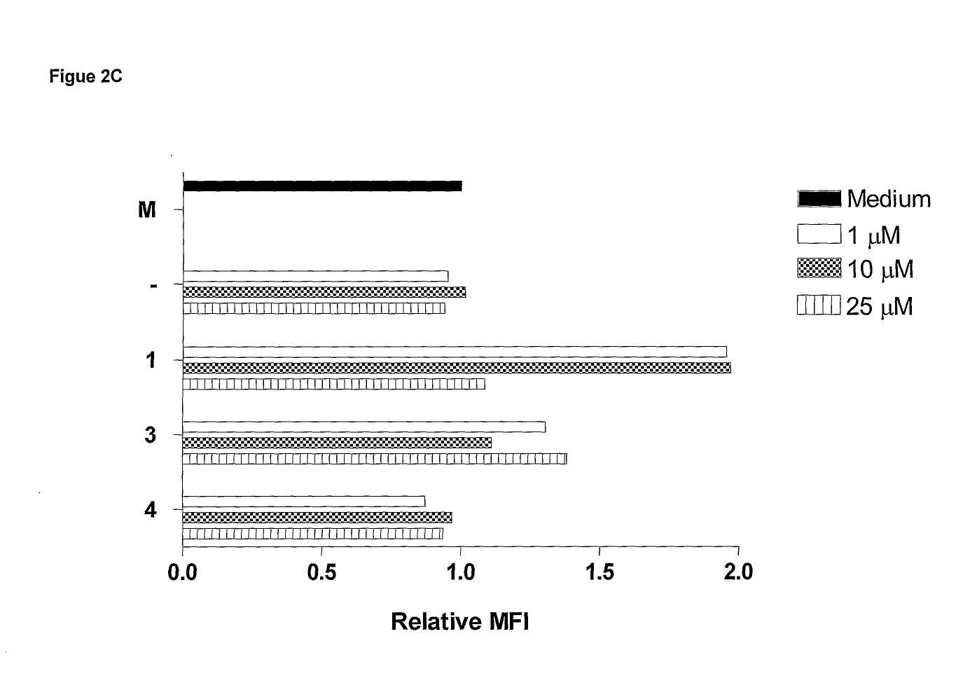

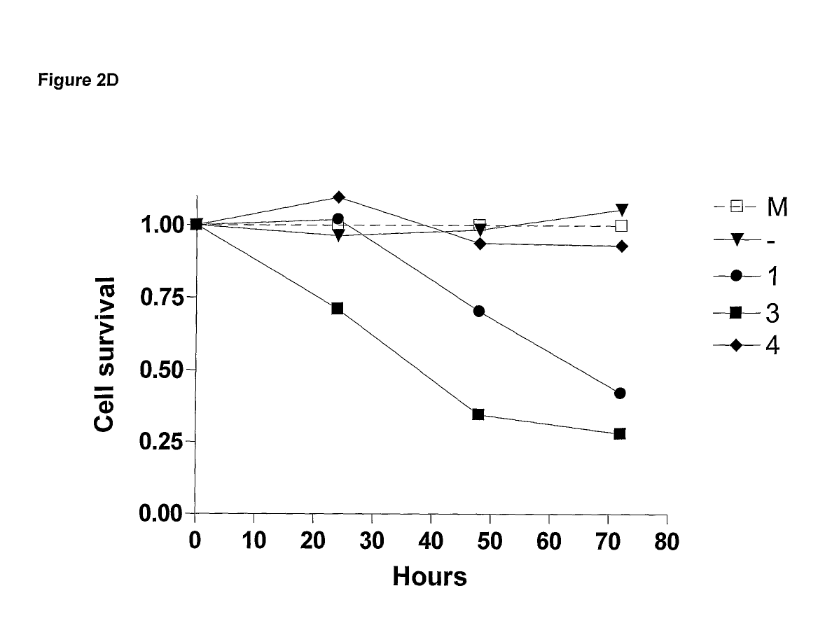

As seen in FIG. 2A, all compounds according to SEQ ID NO. 1-4 were capable of reducing growth of HCT116 tumour cells. In particular, 72 hrs of treatment with SEQ ID NO. 2-4 achieved a marked reduction of tumour growth compared to untreated cells.

FIG. 2B shows the capability of the same compounds to induce apoptosis of HCT116 tumour cells, and here the compounds, in particular SEQ ID NO. 2-4 induced a high rate of apoptosis after 72 hrs of treatment compared to untreated cells. SEQ ID NO. 1 did not induce apoptosis of the HCT116 cell line.

As shown in FIG. 2C, SEQ ID NO. 1 strongly upregulated the cell surface expression of the B-cell proliferation marker CD20 in the Daudi tumour cell line after 48 hrs of treatment. SEQ ID NO. 3 had a modest effect and SEQ ID NO. 4 had no effect on CD20 surface expression.

FIG. 2D shows that 72 hrs of treatment with SEQ ID NO. 1 and 3 resulted in a marked decrease of cell survival of the Daudi cells, whereas SEQ ID NO. 4 had no effect on cell survival of Daudi cells.

3. Cell Surface Receptor Expression in PBMCs Isolated from CLL Blood

### 3.1 Materials and Methods

Heparinized peripheral blood was obtained after informed consent from patients (n=20) diagnosed with B-chronic lymphocytic leukemia (B-CLL) with significant circulating disease. All patients were diagnosed by routine immunophenotypic, morphologic and clinical criteria.

The mononuclear cell fraction was isolated by Ficoll-Hypaque (Seromed, Berlin, Germany) gradient centrifugation. The cells were immediately incubated at 37° C. in a volume of 500 μl of complete RPMI-medium (containing 10% FCS, 1% PenStrep, 2 mM L-glutamine, 10 mM HEPES and 1 mM Sodium Pyruvate) in 48-well plates at a conc. of 2×10⁶ cells/ml and treated with 1, 10 and 25 μM of each of seven different oligonulecleotide compounds. A fraction of the cells were stained with two mixes of 4 antibodies each (CD19, CD20, CD23, CD80 and CD3, CD25, CD56 CD69) for direct analysis of surface antigen expression by FACS.

After 48 hours incubation, 200 μl of the cell suspension was spun down in 96-well plates, resuspended in 100 μl of 2% FCS (in PBS) and incubated with two sets of antibody mixes (as above) for 30 min at 4° C. The cells were then washed twice in pure PBS and subsequently analyzed by FACS using a FACSArray bioanalyzer for surface antigen expression analysis. After 3 days from day 0, the remainder of the cells was harvested for apoptosis analysis. The cells were spun down in 96-well plates, resuspended in 2% FCS as above and incubated with an antibody mix of CD19 and CD3 (BD Pharmingen) for 30 min at 4° C. The cells were washed twice with PBS and subsequently stained with Annexin V and 7-AAD for 10 min at RT for analysis of early and late apoptosis, respectively. The cells were analyzed by flow cytometry as above.

### 3.2 Results

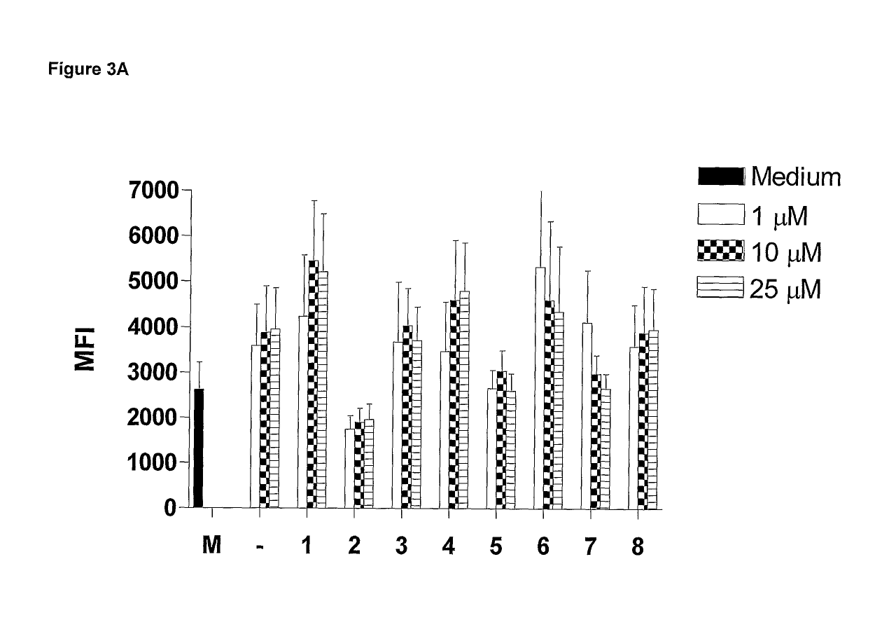

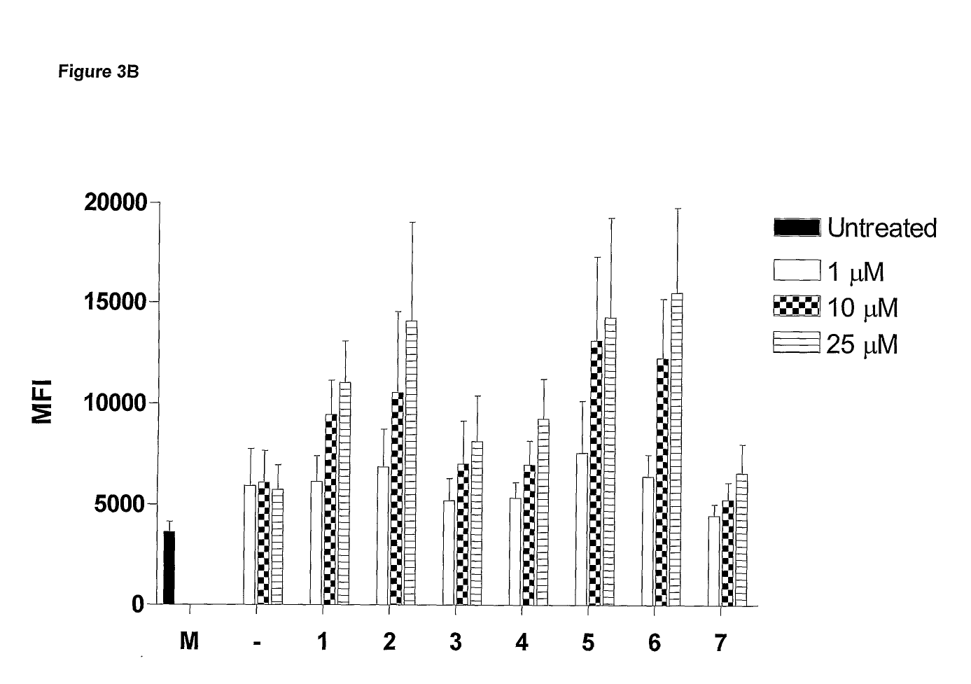

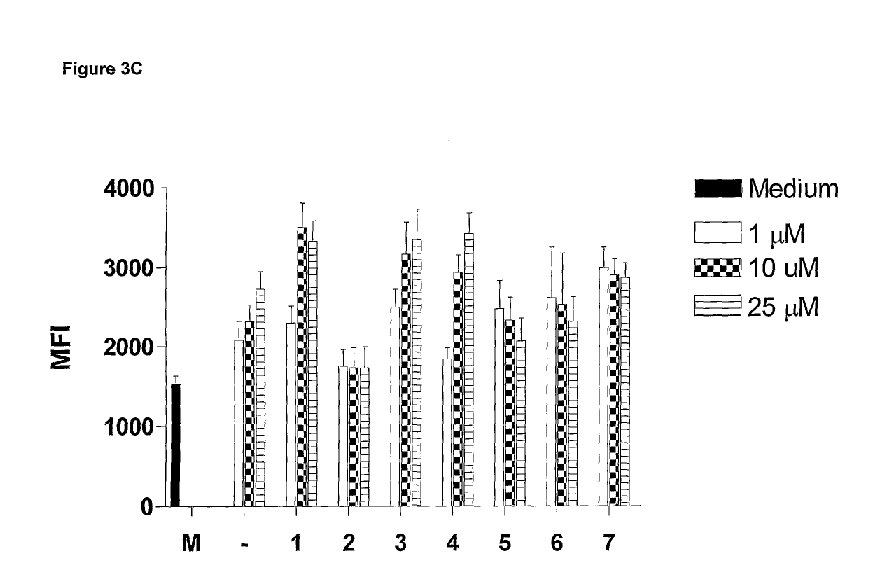

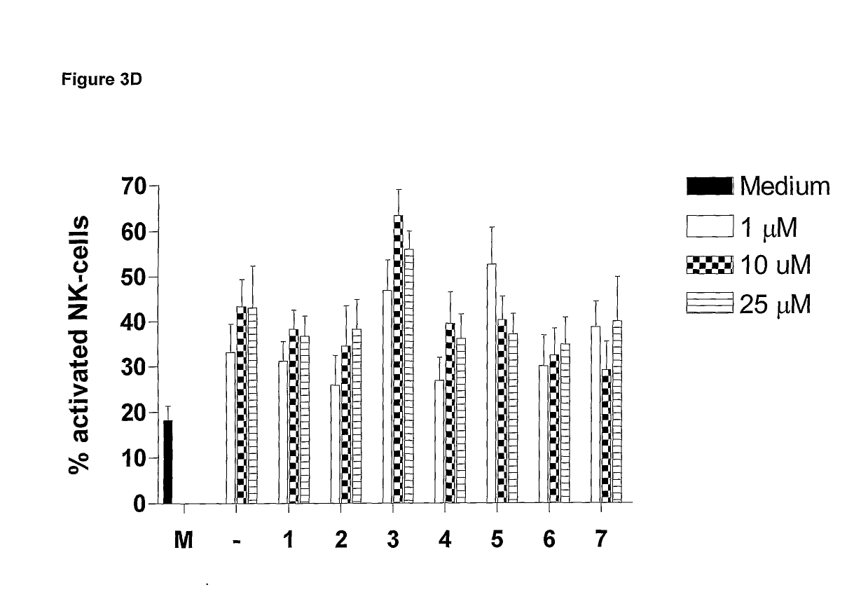

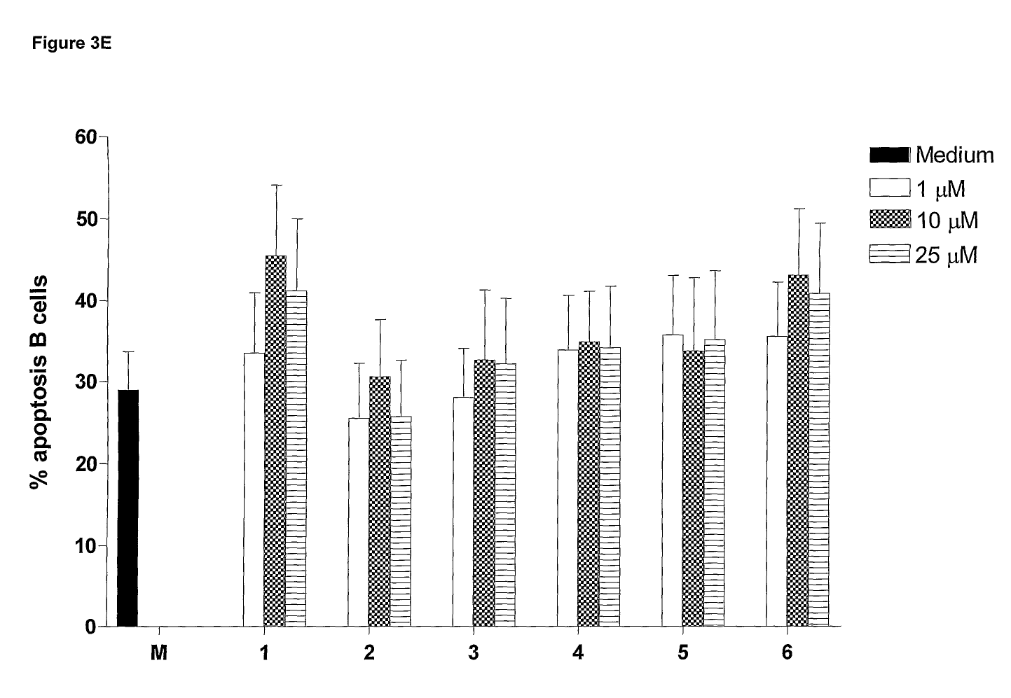

The results show that 48 hrs of treatment with SEQ ID NO. 1, 3, 4, and 6 induced up-regulation of CD20 on B-cells from CLL-patients (FIG. 3A), as well as up-regulation of CD80 on B-cells from CLL-patients (FIG. 3C). SEQ ID NO. 2, 5 and 7 did not upregulate CD20 expression (FIG. 3A) and SEQ ID NO. 2 did not enhance CD80 expression (FIG. 3C).

The expression of CD23 was up-regulated by all SEQ ID NO. (1-7), but most predominantly by SEQ ID NO 1, 2, 5 and 6 (FIG. 3B), with SEQ ID NO 2, 5 and 6 upregulating the receptor heavily.

It was also shown that 48 hrs of treatment with SEQ ID NO 1-7 induce activation of NK-cells as measured by CD69 staining of CD56 positive cells (FIG. 3D).

The results also indicate that SEQ ID NO 1 and 4-6 induce apoptosis of B-cells in PBMCs from CLL-patients (FIG. 3E) after 72 hrs of treatment. SEQ ID NO. 2 and 3 did not induce apotosis of B CLL cells.

### 4. Pulse Experiment

### 4.1 Experimental Setup

The cytokine profile and expression of surface markers was determined in a so called pulse experiment using PBMCs from one healthy volunteer and four CLL patients, respectively. The cytokine profile was determined after 48 hrs cultivation in vitro and the cell surface marker staining was performed by FACS after 72 hrs.

The PBMCs were prepared and cultivated as described in Examples 3 and 4. The PBMCs were then subjected to the SEQ ID NO. 2 for a predetermined period of 30 min, 2 hrs or 6 hrs, followed by washing. The washing was performed as follows: The plates were centrifuged at 1500 rpm for 5 min. Supernatant was discarded and fresh medium was added. Centrifugation was repeated and the second supernatant was replaced by fresh medium. The PBMCs were then cultured further until the desired time points 48 hrs (cytokine profile), or 72 hrs (surface marker staining).

The cytokine profile was determined after 48 hrs in vitro cultivation. Healthy PBMCs were exposed to SEQ ID NO. 1 for the above mentioned timoepoints and the supernatants were analyzed for the contents of IL-6, IL-10, and IP-10. The cytokine concentration is shown as pg/ml.

The surface marker staining was performed after 72 hrs of in vitro cultivation. CLL-PBMCs were treated with SEQ ID NO. 1 for the above mentioned timepoints and the cell surface expression of CD19, CD20, CD56 and CD69 was analyzed by FACS.

### 4.2. Results

The results show that there is a pronounced long term effect even when the oligonucleotide has been removed by washing after only 30 min, which supports the feasibility of nasal administration, or administration to other mucous membranes where the oligonucleotide is not expected to reside for more than about 30 min.

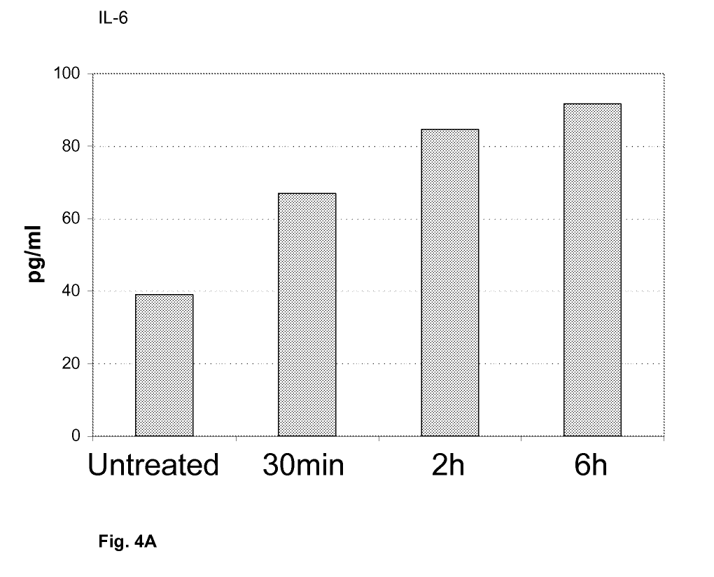

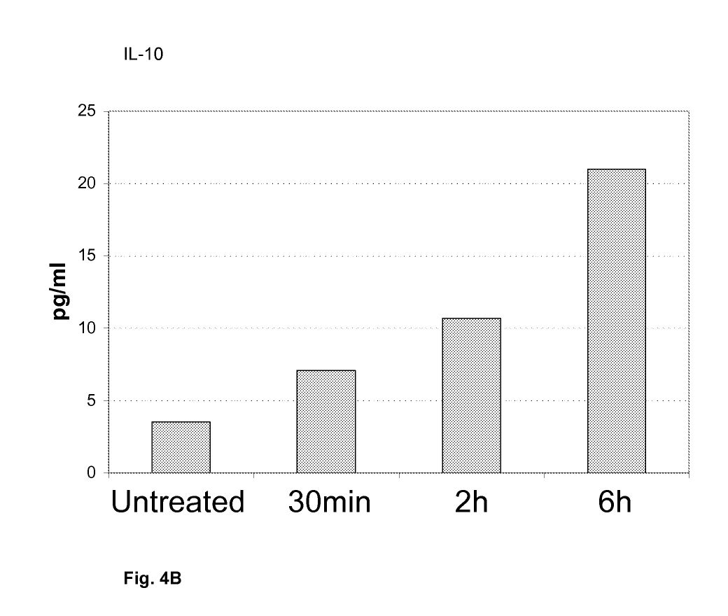

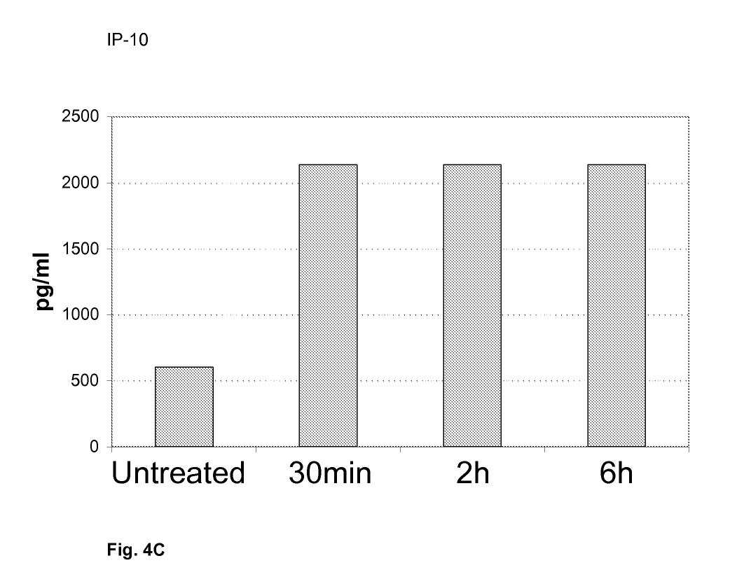

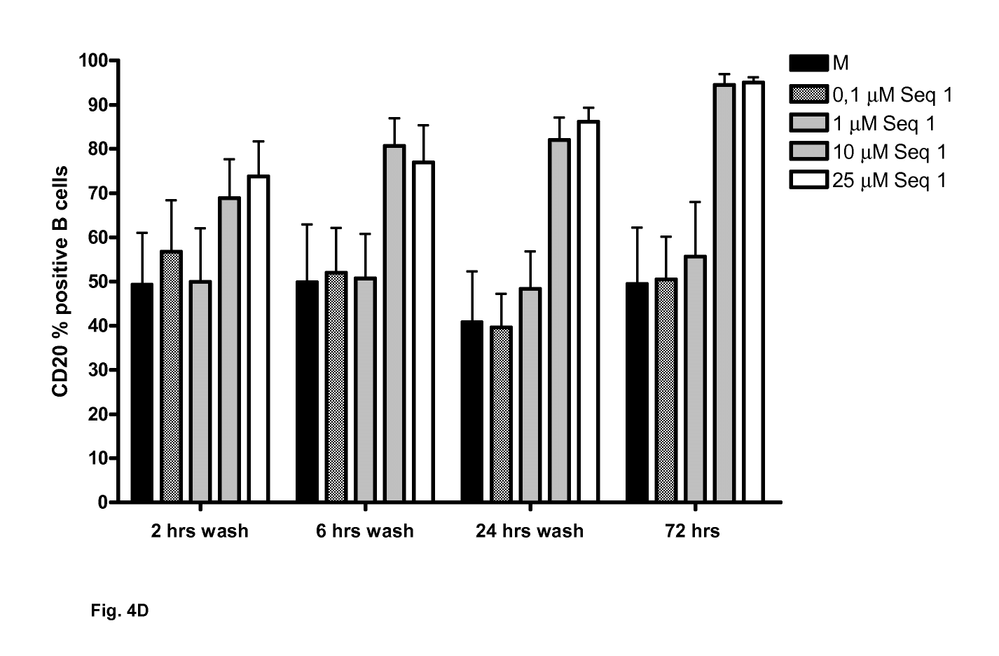

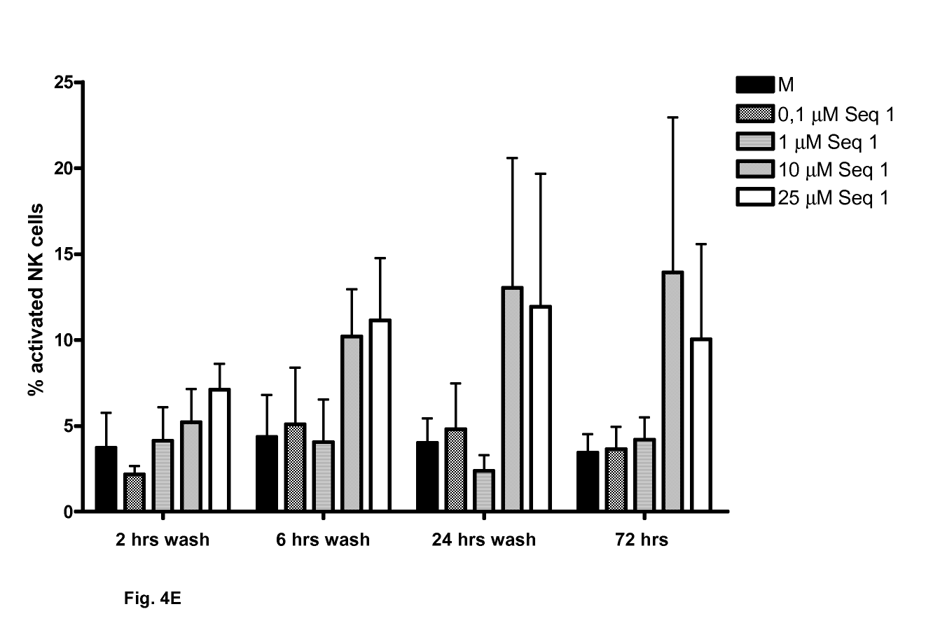

The results also showed a pronounced effect when the oligonucleotide was removed by washing after 2 hrs and also after 6 hrs, corresponding to rectal administration, where a longer residence time is expected. The results are shown in FIGS. 4A, B and C for the cytokine analysis and FIGS. 4D and 4E for the surface marker stainings.

It should also be noted that this experiment was performed using human CLL-PBMCs which makes the results transferable to an in vivo setting with better accuracy than experiments performed with immortalized human cell lines. Notably PBMCs obtained from a diseased patient will contain the malignant B-cells and the effect of the experimental compounds is seen directly on the relevant targets for the therapy.

### 5. Co-Administration of Experimental Compounds and Rituximab

### 5.1 Materials and Methods

Heparinized peripheral blood was obtained after informed consent from patients with B-chronic lymphocytic leukemia (B-CLL). All patients were diagnosed by routine immunophenotypic, morphologic and clinical criteria.

The mononuclear cell fraction was isolated by Ficoll-Hypaque (Seromed, Berlin, Germany) gradient centrifugation. The cells were immediately incubated at 37° C. in a volume of 500 μl of complete RPMI-medium (containing 10% FCS, 1% PenStrep, 2 mM L-glutamine, 10 mM HEPES and 1 mM Sodium Pyruvate) in 48-well plates at a conc. of 2×10⁶ cells/ml.

The cells were incubated with 1, 10 or 25 μM of the experimental compounds, SEQ ID NO. 1, 3, 4, 7 or 8. After 48 hours, the cells were washed twice with PBS and resuspended in complete medium. For the ADCC assay, a CD20 specific monoclonal antibody, rituximab (MabThera®, Roche) was added to a final concentration of 5 μg/ml or 10 μg/ml, together with 10 μg of a F(ab)₂ goat anti-human IgG Fc gamma chain specific antibody (obtained from Jackson Immunoresearch, West Grove, Pa., USA) used as a crosslinker. For the CDC assay, the cells were incubated in 30% human serum (in RPMI) and treated with rituximab for 4 hours after the 48 hour pre-treatment with SEQ ID NO. 1, 3, 4, 7 or 8, and thereafter analysed for apoptosis by flow cytometry. Some cells were treated with rituximab at day 0 for 48 hours and SEQ ID NO. 1 was added day 2 for 24 hours (the reverse experiment).

After 3 days (ADCC) from day 0 (or 2 days and 4 hrs for the CDC assay), cells were harvested for apoptosis analysis. The cells were spun down in 96-well plates, resuspended in 2% FCS as above and incubated with an antibody mix of CD19 and CD3 (BD Pharmingen) for 30 min at 4° C. The cells were washed twice with PBS and subsequently stained with Annexin V and 7-AAD for 10 min at RT for analysis of early and late apoptosis, respectively. The cells were analyzed by flow cytometry as above.

### 5.2 Results

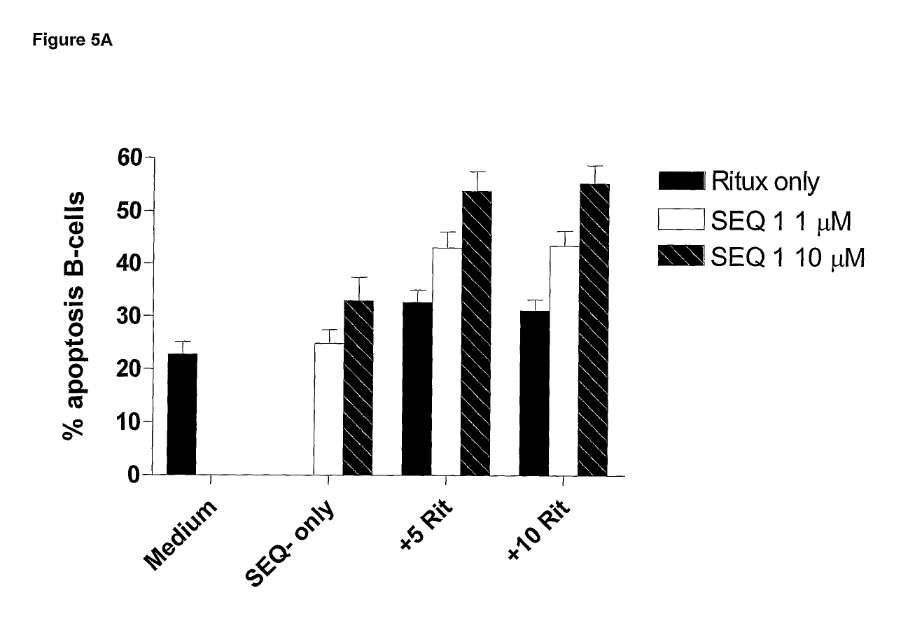

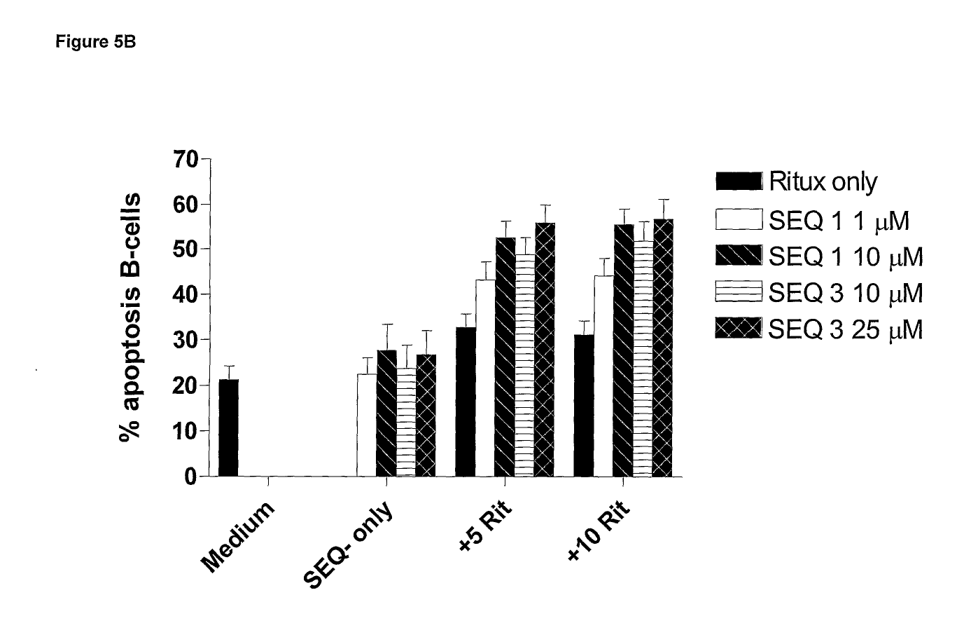

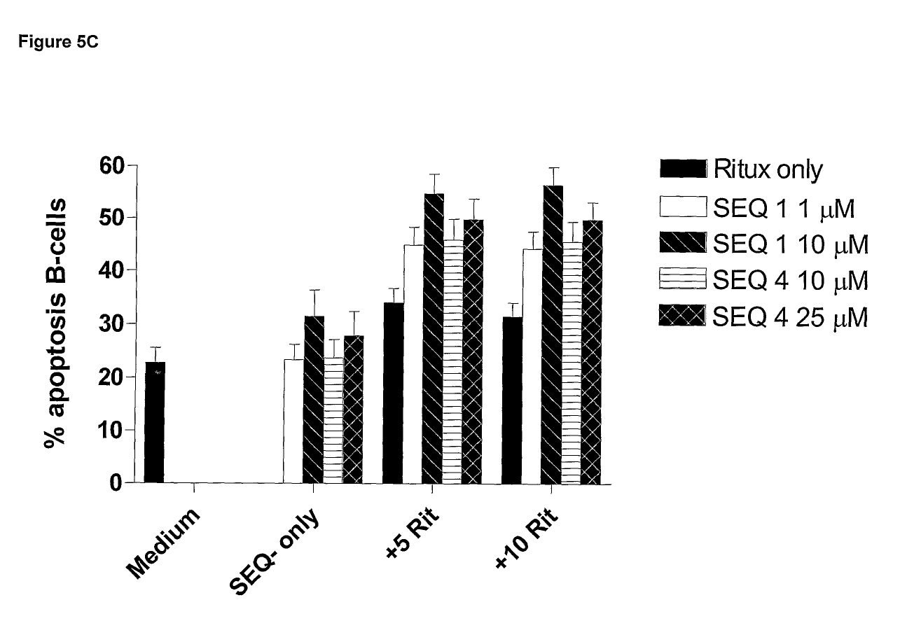

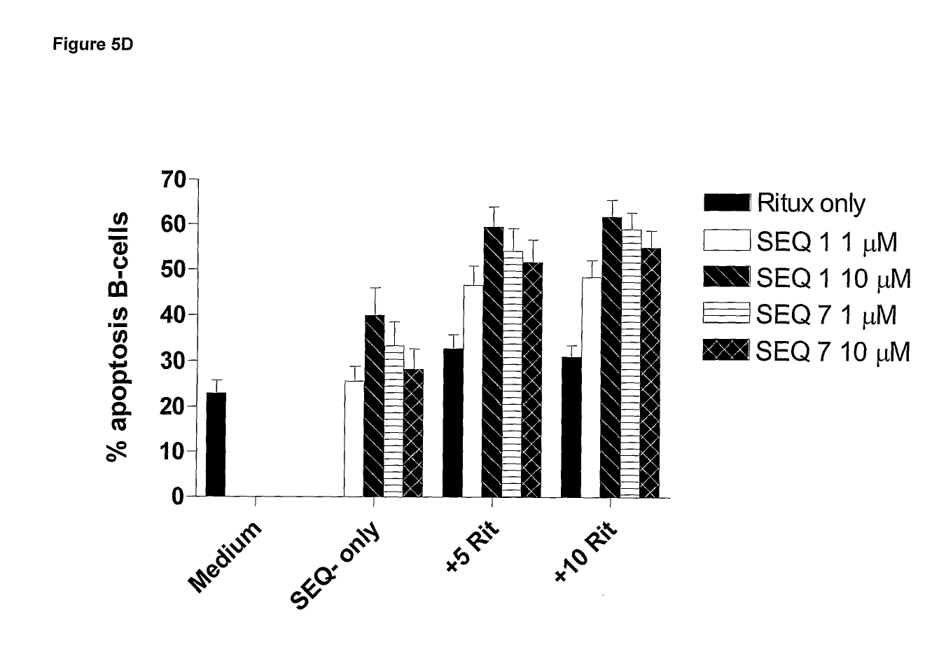

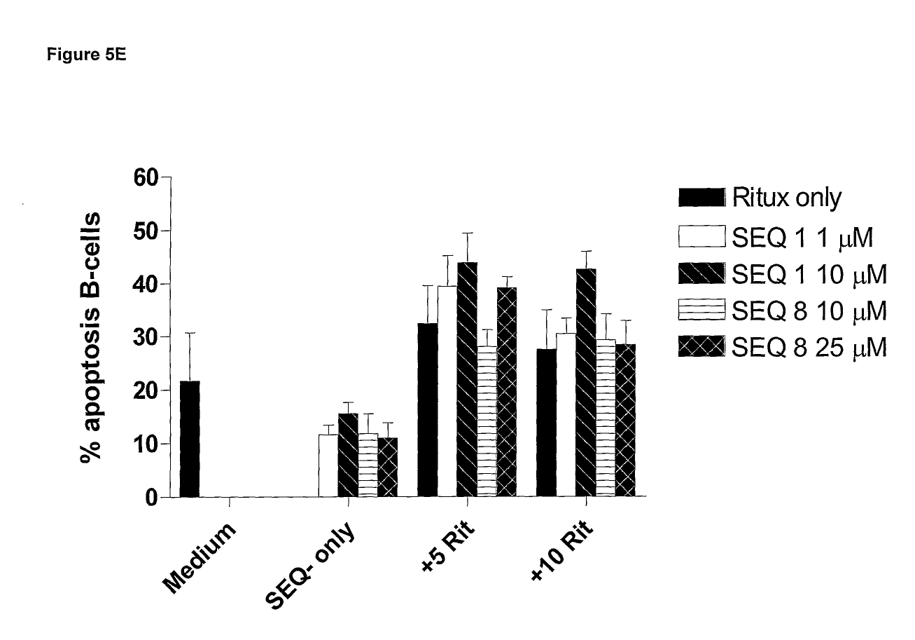

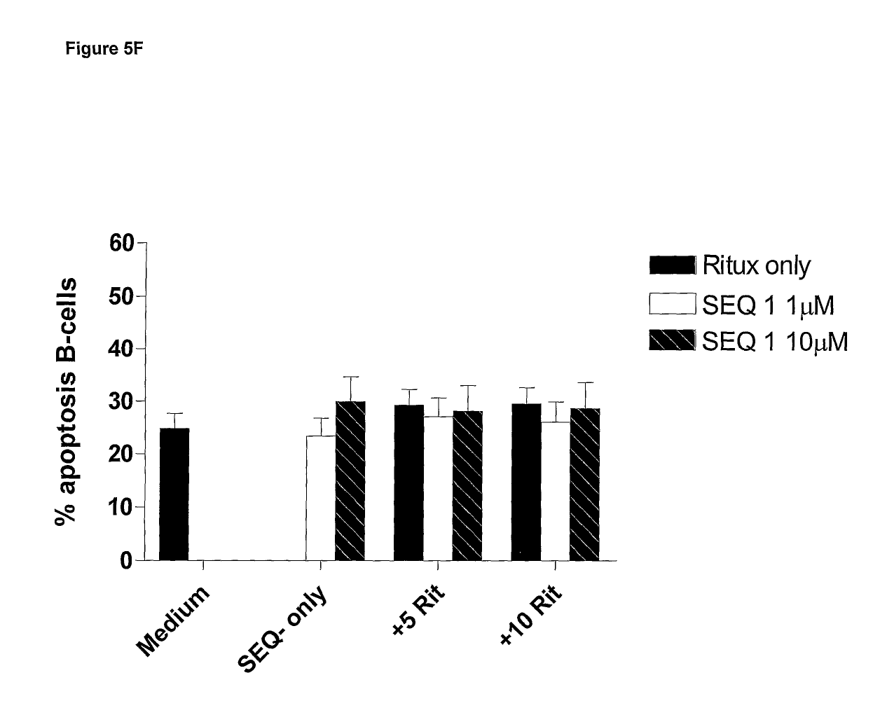

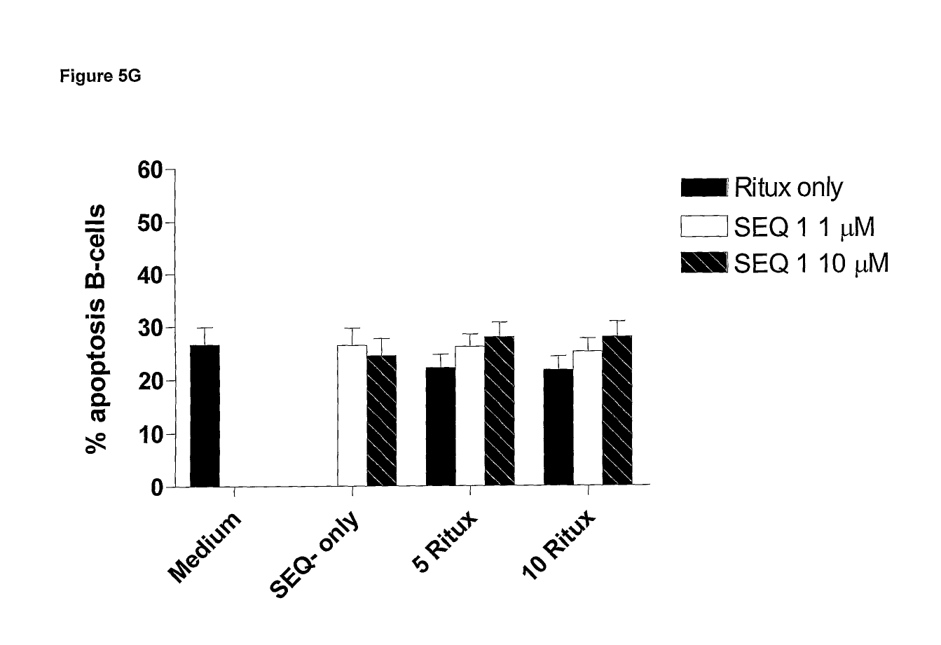

The results clearly show that preincubation with SEQ ID NO. 1 significantly enhanced the efficacy of rituximab-mediated apoptosis of B cells from CLL patients. As mentioned in the background, it is known that rituximab binds human complement and lyses lymphoid B-cell lines through complement-dependent cytotoxicity (CDC). Additionally, rituximab has shown significant activity in assays for antibody-dependent cell-mediated cytotoxicity (ADCC).The results indicate that the combination of SEQ ID NO. 1 and rituximab result in a significantly increased rate of apoptosis of CLL B cells. Pre-treatment with 10 μM of SEQ ID NO. 1 induced a rate of apoptosis almost twice as high to that achieved by rituximab alone (FIG. 5A). Pre-treatment with SEQ ID NO. 3 resulted in an equally effective enhancement of rituximab-mediated apoptosis as pre-treatment with SEQ ID NO. 1 (FIG. 5B). Pre-treatment of CLL-PBMCs using SEQ ID NO. 4 or SEQ ID NO. 7 was not quite as effective (FIGS. 5C and D), while pre-treatment of cells with SEQ ID NO. 8 had no effect on rituximab-induced cell death (FIG. 5E). The observed increase in apoptosis was only seen in the ADCC assay (FIG. 5A), while no effect was observed in the CDC assay (FIG. 5F and data not shown).

Further, the experiments indicate that the order of administration is important. As shown in FIG. 5A, prior administration of SEQ ID NO. 1 significantly enhanced rituximab-mediated apoptosis of B cells, while the reverse experiment (i.e. cells were first treated with rituximab and SEQ ID NO. 1 was added after 48 hrs of rituximab treatment) did not result in an increase in apoptosis compared to cells treated with rituximab alone, see FIG. 5G.

6. Cytokine Analysis of Cells Treated with Experimental Compounds and Rituximab

### 6.1 Materials and Methods

PBMCs isolated from CLL blood were treated with 1, 10 and 25 μM of SEQ ID NO. 1-6. After 48 hrs of treatment, supernatants were harvested and analyzed for cytokine content by CBA. Analysis was performed to investigate differences between different CLL samples.

### 6.2 Results

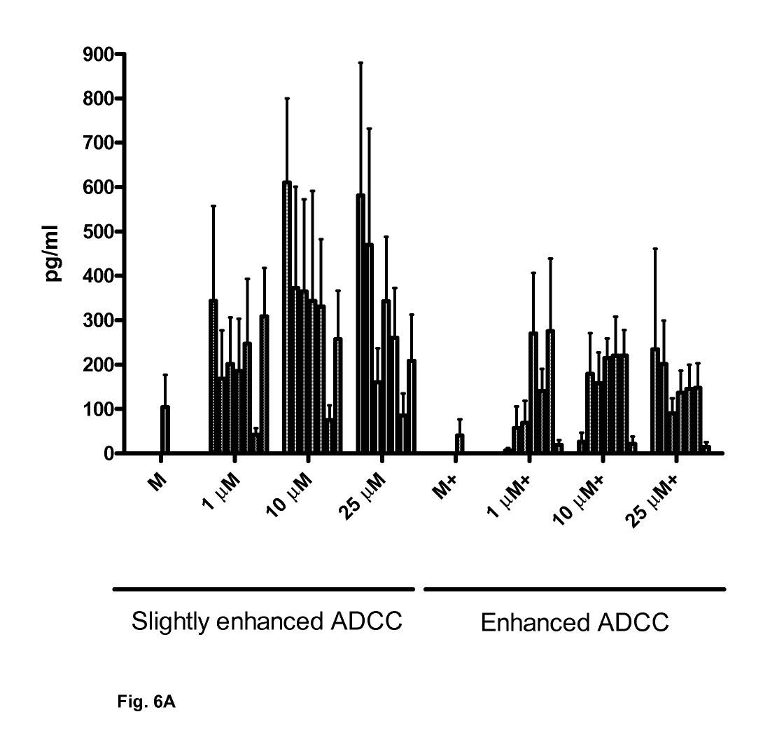

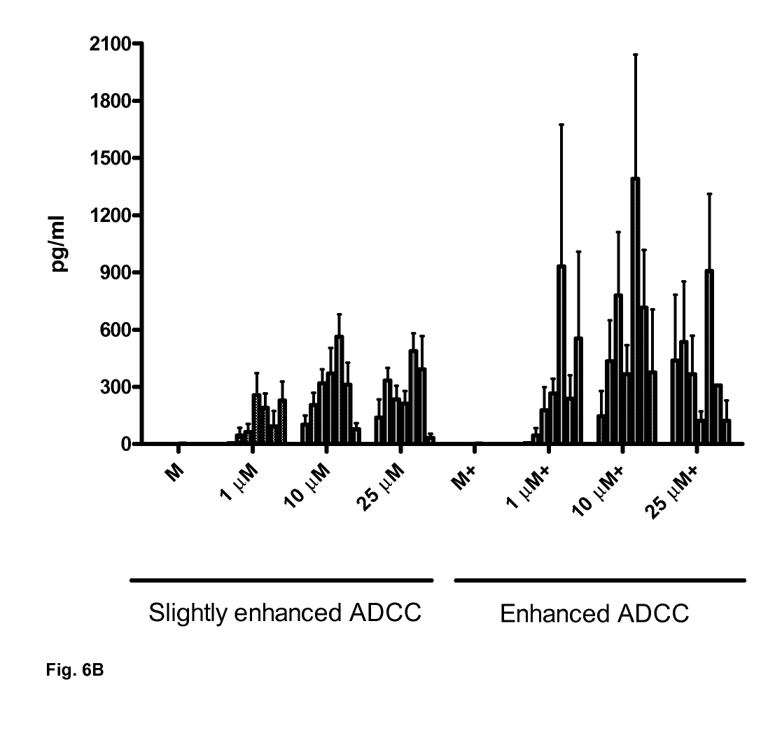

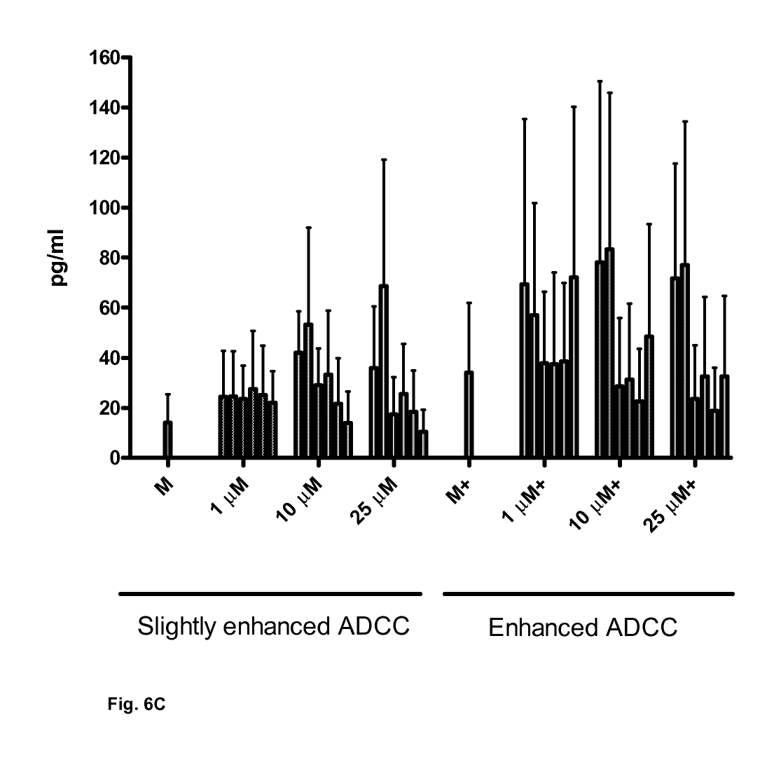

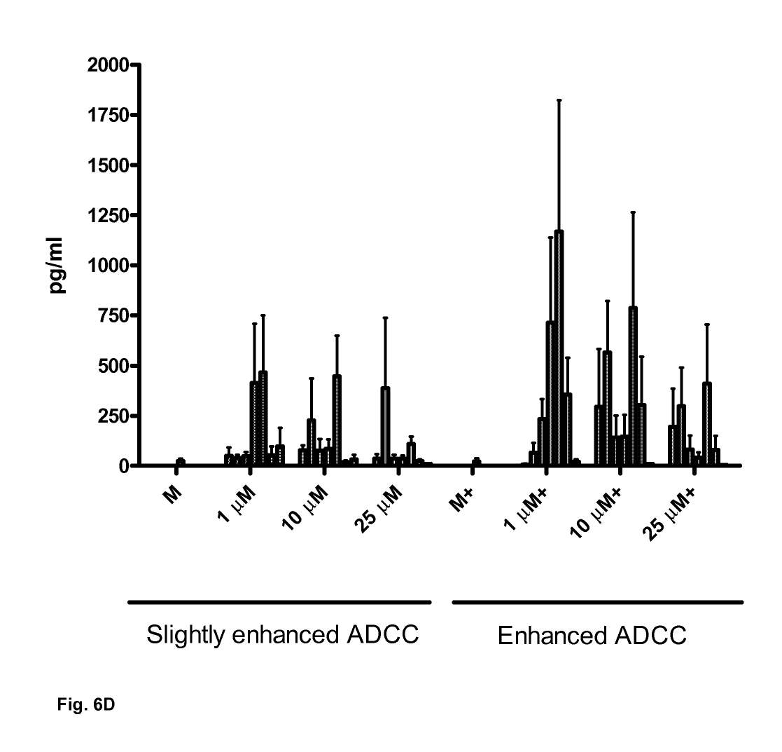

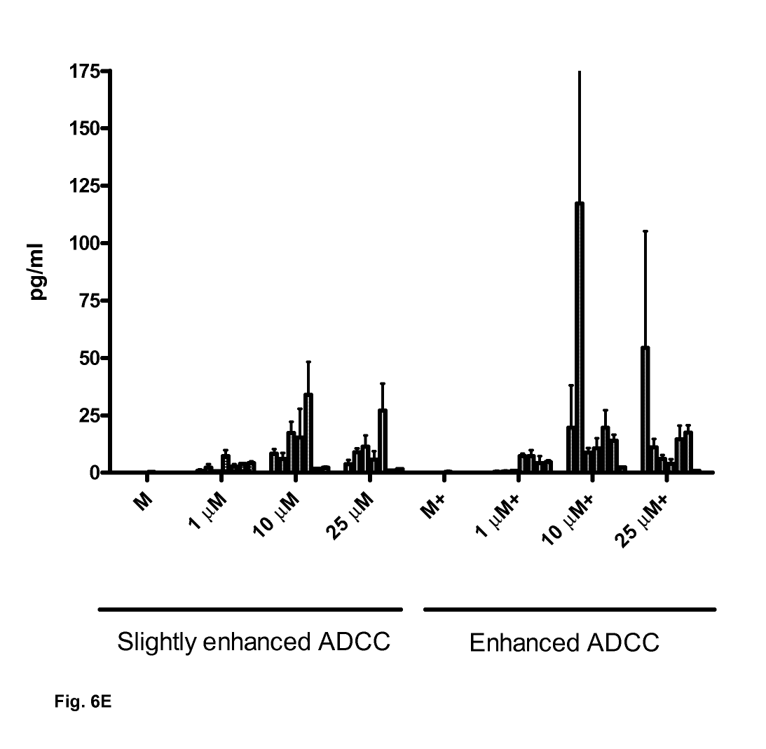

The results show that CLL samples responding well to combination treatment with experimental compounds and rituximab, expressed higher amounts of Th1-like cytokines compared to samples responding less well to combination treatment. As seen in FIG. 6A, samples responding well to combination treatment produce less amounts of IL-6 compared to non-responding cells. On the other hand, responding cells produced more of IL-10 (FIG. 6B), IL-12 (FIG. 6C), IP-10 (FIG. 6D) and TNF-α (FIG. 6E). There was no difference in the expression of G-CSF (data not shown).

In summary, the present invention describes the oligonucleotide induced modulation of cell surface receptors leading to enhanced efficacy of antibody based therapy used for treating chronic lymphocytic leukaemia. The investigated compounds were initially chosen based on their respective patterns of cytokine induction in healthy PBMCs. Surprisingly, when used for analyzing the effects on surface antigens expressed on CLL cells, the inventors found that not all compounds upregulated all receptors, but instead, certain compounds upregulated certain receptors. For instance, SEQ ID NO. 1 was the most potent upregulator of the cell surface markers CD20 and CD80, while SEQ ID NO. 6 was the most potent upregulator of CD23. SEQ ID NO. 3 was the strongest activator of NK cells as shown by a strong upregulation of CD69 on NK cells. Combination treatment of CLL-PBMCs with SEQ ID NO. 1 and rituximab resulted in a significant increase of rituximab-mediated ADCC as compared to rituximab used alone. As indicated by their varying abilities in upregulating CD20, different compounds had different abilities in enhancing rituximab-induced ADCC. Surprisingly though, there was no increase in cell death mediated through the complement system. This could be of importance for the induction of side-effects, where activation of the complement system is regarded as being more toxic to a patient than activation of ADCC. Taken together, the results indicate that the inventive compounds enhance the efficacy of monoclonal antibody therapies designed to treat CLL, where specific compounds could be used in combination with specific antibodies.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

### REFERENCES

U.S. Pat. No. 6,498,147 B (THE SCRIPPS INSTITUTE) 24 Dec. 2002

Krieg, A M. Lymphocyte activation by CpG dinucleotide motifs in prokaryotic DNA. Trends Microbiol. 1996, vol. 4, no. 2, p. 73-6.

Krieg, A M, et al. Mechanisms and therapeutic applications of immune stimulatory cpG DNA. Pharmacol Ther. 1999, vol. 84, no. 2, p. 113-20.

Wooldridge, J E, et al. CpG DNA and cancer immunotherapy: orchestrating the antitumour immune response. Curr Opin Oncol. 2003 November, vol. 15, no. 6, p. 440-5.

Caligaris-Cappio, F., Hamblin, T. J.: Bal1 chronic lymphocytic leukemia: a bird of a different feather. J Clin Oncol. 1999 17:399406.

Rozman, C., Montserrat, E.: Chronic lymphocytic leukemia. New Engl J Med. 1995 333: 1052-1059.

Gaidan, G. L., Ballerini, P., Gong J Z, et al: p53 mutations in human lymphoid malignancies: association with Burkitt lymphoma and chronic lymphocytic leukemia. Proc Nat Acad Sci USA. 1991 88:541 3-541 8.

Dohner, H., Stilgenbauer, S., Doner, K., Bentz, M., Lichter, P.: Chromosome aberration in B-cell chronic lymphocytic leukemia: reassessment based on molecular genetic analysis. J Mol Med. 1999 77:266-270.

Osorio, L. M., Jondal, M. Aguilar-Santelises, M.; Regulation of BCLL Apoptosis Through Membrane Receptors and Bcl-2 Family Proteins; Leukemia and Lymphoma. 1999 30:247-256.

Dancescu, M., Rubio-Trujillo M, Biron G, Bron D, Delespesse G, Sarfati M. Interleukin 4 protects chronic lymphocytic leukemic B cells from death by apoptosis and upregulates Bcl-2 expression. J Exp Med. 1992 Nov. 1; 176(5):1319-26.

Klein, A, Miera, O., Bauer, O., Golfier, S., Schriever. F.; Chemosensitivity of 8 cell chronic lyrnphocytic leukemia and correlated expression of proteins regulating apoptosis, cell cycle and DNA repair; Leukemia. 2000 14.40-46.

Pistoia, V.: Production of cytokines by human B cells in health and disease. Immunol Today. 1997 18:343-346.

Tangye, S. G., Weston, K. M., Raison, R. L.; Interleukin-10 Inhibits the In vitro Proliferation of Human Activated Leukemic CD5+ B-cells; Leukemia and Lymphoma. 1999 30: 121-130.

Strome S. E, Sausville E A, Mann D. A mechanistic perspective of monoclonal antibodies in cancer therapy beyond target-related effects. Oncologist 2007, 12(9):1084-95.

Pescovitz, M. D. Rituximab, an Anti-CD20 Monoclonal Antibody: History and Mechanism of Action. Am J Transpl. 2006 (6): 859-866.

Reff, M. E., Garner K., Chambers K. S., Chinn P. C., Leonard J. E., Raab R., Newman R. A., Hanna N., Anderson D. R. Depletion of B cells in vivo by a chimeric mouse human monoclonal antibody to CD20. Blood. 1994 Jan. 15; 83(2):435-45.

Leonard, John P., LINK Brian K., EMMANOUILIDES Christos, GREGORY Stephanie A., WEISDORF Daniel, ANDREY Jeffrey, HAINSWORTH, John, SPARANO Joseph A., TSAI Donald E., HORNING Sandra, KRIEG Arthur M. and WEINER George J., Phase I Trial of Toll-Like Receptor 9 Agonist PF-3512676 with and Following Rituximab in Patients with Recurrent Indolent and Aggressive Non-Hodgkin's Lymphoma, in Clinical Cancer Research 13, 6168-6174, Oct. 15, 2007.

Pathan, N. I., Chu P., Hariharan K., Cheney C., Molina A., Byrd J. Mediation of apoptosis by and antitumour activity of lumiliximab in chronic lymphocytic leukemia cells and CD23+ lymphoma cell lines. Blood. 2008 Feb. 1; 111(3):1594-602.

Keating M. J. Management of chronic lymphocytic leukemia: a changing field. Rev Clin Exp Hematol 2002, 6(4):350-65

Krieg A. M, Yi A K, Matson S, Waldschmidt T J, Bishop G A, Teasdale R, Koretzky G A, Klinman D M. CpG motifs in bacterial DNA trigger direct B-cell activation. Nature. 1995, Apr. 6; 374 (6522):546-9.

Sokoloski, J A, et al. Antisense oligonucleotides to the p65 subunit of NF-kB block CD11b expression and alter adhesion properties of differentiated HL-60 granulocytes. Blood. 15 Jul. 1993, vol. 82, no. 2, p. 625-632.

Gursel, et al. Differential and competitive activation of human immune cells by distinct classes of CpG oligodeoxynucleotide. J Leuk Biol. 2002, vol. 71, p. 813-820.

Jahrsdorfer, et al. CpG DNA increases primary malignant B cell expression of costimulatory molecules and target antigens. J Leuk Biol. 2001, vol. 69, p. 81-88.

Jahrsdorfer, et al B-cell lymphomas differ in their responsiveness to CpG oligodeoxynucleotides. Clin Can Res. 2005a, vol. 11, p. 1490-1499.

Jahrsdorfer, et al. Immunostimulatory oligodeoxynucleotides induce apoptosis of B cell chronic lymphocytic leukemia cells. J Leuk Biol. 2005b, vol. 77, p. 378-387.

1.-22. (canceled)

23. An isolated oligonucleotide sequence according to any one of SEQ ID NO. 1-3 and 5-7.

24. An isolated oligonucleotide sequence according to claim 23, wherein at least one nucleotide has a phosphate backbone modification.

25. A medicament for the treatment of cancer comprising an isolated oligonucleotide sequence according to any one of SEQ ID NO. 1-3 and 5-7.

26. A medicament for the induction of apoptosis comprising an isolated oligonucleotide sequence according to any one of SEQ ID NO. 1-7.

27. A medicament for up-regulating the expression of a cell surface antigen chosen from CD20, CD23, CD69 and CD80, comprising an isolated oligonucleotide sequence according to any one of SEQ ID NO. 1-7.

28. A medicament according to claim 27, for up-regulating the expression of CD20, comprising an isolated oligonucleotide sequence according to SEQ ID NO. 1, SEQ ID NO. 4 or SEQ ID NO. 6.

29. The medicament according to claim 27, wherein the medicament is adapted to be administered topically to a mucous membrane or subcutaneously in a dose effective to up-regulate the expression of at least one of the cell surface markers CD20, CD23, CD69, and CD80.

30. The medicament according to claim 29, wherein the dose is in the interval of about 0.01 to about 50 mg/kg body weight, more preferably 0.05 to about 5 mg/kg body weight and most preferably 0.1 to about 1 mg/kg body weight.

31. The medicament according to claim 25, wherein said at least one oligonucleotide has a phosphate backbone modification.

32. A method for the treatment of cancer, wherein an isolated oligonucleotide sequence according to any one of SEQ ID NO. 1-3 and 5-7 is administered to a patient in need thereof.

33. The method according to claim 32, wherein said oligonucleotide is administered topically to a mucous membrane of a patient in need thereof.

34. The method according to claim 32, wherein said oligonucleotide is administered subcutaneously to a patient in need thereof.

35. The method according to claim 32, wherein said at least one oligonucleotide has a phosphate backbone modification.

36. The method according to claim 32, wherein said oligonucleotide is administered in a dose of about 0.01 to about 50 mg/kg body weight, more preferably 0.05 to about 5 mg/kg body weight and most preferably 0.1 to about 1 mg/kg body weight.

37. The method according claim 32, wherein said oligonucleotide is administered before or essentially simultaneously with an anti-tumour treatment.

38. The method according to claim 37, wherein the anti-tumour treatment is chosen among radiation treatment, hormone treatment, surgical removal of the tumour, chemotherapy, immunological or immunomodulating therapy, photodynamic therapy, laser therapy, hyperthermia, cryotherapy, angiogenesis inhibition, or a combination of any of these.

39. The method according to claim 37, wherein said anti-tumour treatment is an immunological treatment and comprises the administration of an antibody to the patient.

40. The method according to claim 37, wherein said oligonucleotide sequence is administered to a patient before the administration of an antibody.

41. A method for the treatment of cancer, wherein an oligonucleotide sequence chosen among SEQ ID NO. 1-7, is administered in a dose effective to elicit the expression of at least one of the cell surface markers CD20, CD23, CD69 and CD80.

42. The method according to claim 41, wherein said at least one oligonucleotide has a phosphate backbone modification.

43. The method according to claim 41, wherein said oligonucleotide is administered in a dose of about 0.01 to about 50 mg/kg body weight, more preferably 0.05 to about 5 mg/kg body weight and most preferably 0.1 to about 1 mg/kg body weight.

44. The method according claim 41, wherein said oligonucleotide is administered before or essentially simultaneously with an anti-tumour treatment.

45. The method according to claim 44, wherein the anti-tumour treatment is chosen among radiation treatment, hormone treatment, surgical removal of the tumour, chemotherapy, immunological or immunomodulating therapy, photodynamic therapy, laser therapy, hyperthermia, cryotherapy, angiogenesis inhibition, or a combination of any of these.

46. The method according to claim 44, wherein said anti-tumour treatment is an immunological treatment and comprises the administration of an antibody to the patient.

47. The method according to claim 44, wherein said oligonucleotide sequence is administered to a patient before the administration of an antibody.


SEQUENCE LISTING (8 sequences)

SEQ ID NO: 1 - DNA, 22 bp, Artificial Sequence
  Synthetic construct
tcgtcgttctgccatcgtcgtt

SEQ ID NO: 2 - DNA, 14 bp, Artificial Sequence
  Synthetic construct
ggggtcgtctgcgg

SEQ ID NO: 3 - DNA, 16 bp, Artificial Sequence
  Synthetic construct
gatcgtccgtcggggg

SEQ ID NO: 4 - DNA, 19 bp, Artificial Sequence
  Synthetic construct
ggaacagttcgtccatggc

SEQ ID NO: 5 - DNA, 21 bp, Artificial Sequence
  Synthetic construct
tcgtcgttcggccgatcgtcc

SEQ ID NO: 6 - DNA, 21 bp, Artificial Sequence
  Synthetic construct
tcgttcgtctgcttgttcgtc

SEQ ID NO: 7 - DNA, 19 bp, Artificial Sequence
  Synthetic construct
ggaacagttgctccatggc

SEQ ID NO: 8 - DNA, 24 bp, Artificial Sequence
  Synthetic construct
ccggggtcgcagctgagcccacgg